(19) United States
(12) Patent Application Publication
(10) Pub. No.: US 2026/0144783 A1
(43) Pub. Date: May 28, 2026

(54) COMPOSITION FOR PREVENTING OR TREATING PERIPHERAL NEUROPATHY, CONTAINING ISOQUINOLINE DERIVATIVE AS ACTIVE INGREDIENT

(71) Applicants: ALTMEDICAL CO., LTD., Seoul (KR); DONG-A UNIVERSITY RESEARCH FOUNDATION FOR INDUSTRY-ACADEMY COOPERATION, Busan (KR)

(72) Inventors: Jeanho YUN, Busan (KR); Jong Hyun CHO, Gyeongsangnam-do (KR); Ji Hyun LEE, Busan (KR); Sangwoo IM, Busan (KR); Se Myeong CHOI, Busan (KR)

(21) Appl. No.: 19/120,941

(22) PCT Filed: Oct. 12, 2023
(86) PCT No.: PCT/KR2023/015689

§ 371 Date: Apr. 14, 2025

(30) Foreign Application Priority Data
Oct. 14, 2022 (KR) 10-2022-0132390

(51) Int. Cl. A61K 31/4745; A61K 9/00; A61K 9/08; A61K 9/20; A61K 9/48; A61K 47/02; A61K 47/14; A61K 47/46; A61P 25/02

(52) U.S. Cl. CPC A61K 31/4745; A61K 9/0019; A61K 9/0043; A61K 9/0056; A61K 9/08; A61K 9/2013; A61K 9/2018; A61K 9/2059; A61K 9/4825; A61K 9/4858; A61K 9/4866; A61K 47/02; A61K 47/14; A61K 47/46; A61P 25/02

(57) ABSTRACT

The present invention relates to a pharmaceutical composition for preventing or treating peripheral neuropathy, and the like. The present invention has been completed by confirming that an isoquinoline derivative discovered through a screening based on mitophage activity exhibits an excellent mitophage promoting effect, and thus, the isoquinoline derivative can be utilized as a fundamental therapeutic agent for peripheral neuropathy. Specifically, it has been found that the isoquinoline derivative according to the present invention treats hyperalgesia in an animal model of anticancer drug-inducible peripheral neuropathy, also suppresses morphological changes of sensory nerves, which have been identified as a key cause of peripheral neuropathy, and can increase the distribution and number of peripheral nerves. Therefore, the isoquinoline derivative according to the present invention is a fundamental therapeutic agent capable of suppressing the main symptoms and causes of peripheral neuropathy, particularly, anticancer drug-induced peripheral neuropathy, and is expected to be usefully utilized in the field of prevention, amelioration, and/or treatment of the disease.

### TECHNICAL FIELD

The present invention relates to uses of a novel isoquinoline derivative to prevent, improve, and/or treat peripheral neuropathy.

This application claims priority to and the benefit of Korean Patent Application No. 10-2022-0132390, filed on Oct. 14, 2022, the disclosure of which is incorporated herein by reference in its entirety.

### BACKGROUND ART

Peripheral neuropathy refers to a disorder in which various impairments in bodily functions occur due to damage to the peripheral nervous system that is distributed throughout the body, including the hands and feet. Peripheral neuropathy is caused by diseases that directly or indirectly affect nerve tissues, and it may be classified into sensory neuropathy, motor neuropathy, or autonomic neuropathy depending on the type of nerve tissue affected. In the case of sensory neuropathy, patients may experience decreased sensitivity to tactile stimuli or temperature changes, tingling sensations or burning pain, and allodynia in the skin. Motor neuropathy is accompanied by impaired balance or muscle weakness, while autonomic neuropathy may cause weakened bladder control leading to urinary incontinence or may induce abnormal blood pressure and heart rate, depending on the organs innervated by the affected nerves. Various factors have been proposed as causes of peripheral neuropathy, and it is known that genetic disorders, nutritional deficiencies, metabolic and endocrine diseases, diabetes, inflammatory diseases, and administration of anticancer agents for cancer treatment are among the causes. Among these, chemotherapy-induced peripheral neuropathy (CIPN) is a representative form of acquired peripheral neuropathy, occurring in more than 60% of patients undergoing chemotherapy, and causes severe symptoms such as sensory abnormalities, allodynia, and hyperalgesia. CIPN has a significant impact on the treatment and quality of life of cancer patients. However, since there is currently no therapeutic agent available for peripheral neuropathy, the only approaches being attempted are dose reduction of the chemotherapeutic agent or switching to another anticancer drug.

According to previous studies, dysfunction of axonal mitochondria has been identified as a cause of chemotherapy-induced peripheral neuropathy (CIPN) induced by chemotherapeutic agents such as paclitaxel, vincristine, taxol, cisplatin, and bortezomib. In particular, upon treatment with paclitaxel, phenomena such as a reduction in mitochondrial membrane potential, an increase in mitochondrial reactive oxygen species (ROS), a decrease in ATP synthesis, and structural abnormalities in mitochondria have been observed in both cellular and animal models. Considering that mitochondria in neurons play essential roles in energy production, neuroplasticity, and resistance to stress, mitochondrial dysfunction induced by chemotherapeutic agents is believed to play a crucial role in the onset and progression of CIPN. However, to date, strategies for treating CIPN by improving mitochondrial dysfunction have not yet been developed.

Meanwhile, mitophagy is an intracellular degradation mechanism that removes damaged or unnecessary mitochondria. When mitochondria are damaged, they are surrounded by a membrane to form an autophagosome, which is then fused with a lysosome to selectively remove the damaged mitochondria. It is known that the activity of the mitophagy is important for regulating mitochondria function in various cells including neurons and maintaining tissue functions. In addition, mitophagy in neurons has been reported to have a protective effect against various stresses and to be important for resistance to neurodegeneration. Recently, decreased mitophagy activity has been observed in neurodegenerative diseases such as Alzheimer's disease and Parkinson's disease, and experimental studies using animal models of Alzheimer's disease or Parkinson's disease have demonstrated that promoting mitophagy improves mitochondrial dysfunction and alleviates pathological symptoms. Therefore, in chemotherapy-induced peripheral neuropathy (CIPN), which is caused by degeneration of neuronal cells, promoting mitophagy is expected to be a promising strategy for improving mitochondrial dysfunction and treating symptoms such as hyperalgesia. However, therapeutic strategies for treating CIPN through the promotion of mitophagy have not yet been attempted due to a technical limitation, namely, the absence of substances capable of effectively promoting mitophagy.

In order to discover a mitophagy regulator that can exert a disease treatment effect by promoting mitophagy activity, it is essential to have a research system that can easily measure mitophagy changes in cells and in vivo. However, due to the absence of experimental methods for sensitively and quantitatively measuring mitophagy activity, searching for a mitophagy regulating compound using mitophagy activity itself as a read-out has not been performed. The LC3-based detection method that has been widely used so far has limitations in that it can only measure an autophagosome-forming stage, which is the initial stage of mitophagy, and therefore has low measurement sensitivity and difficulty in quantitative measurement. In addition, most attempts to discover control materials in several research groups have also failed to discover materials that can control mitophagy activity under actual physiological conditions because they used indirect read-out indicators such as the mitochondrial movement of PINK-Parkin or the mitochondrial fission that occurs during mitophagy.

Currently, experimental methods to induce mitophagy activity involve treating with so-called ‘mitochondrial toxins’, such as CCCP, FCCP, and rotenone, which induce mitochondrial dysfunction. However, the CCCP and FCCP depolarize the mitochondrial membrane potential as a mitochondrial membrane potential inhibitors (uncouplers), and rotenone acts as a Complex I inhibitor. These mitochondrial toxins induce mitophagy activity by directly causing mitochondrial damage, thereby activating the mechanism for removing damaged mitochondria. However, their strong toxicity to cells limits their use as drugs to promote mitophagy activity.

### DISCLOSURE

### Technical Problem

As described above, chemotherapeutic agents, including paclitaxel, frequently induce peripheral neuropathy that causes pain and sensory abnormalities: however, due to the absence of effective therapeutic agents therefor, they have been identified as a major cause of reduced quality of life in cancer patients. The present invention has been devised to solve the above-mentioned problems, and was completed by confirming that an isoquinoline derivative identified through screening based on mitophagy activity may improve and alleviate major symptoms of peripheral neuropathy by promoting mitophagy activity, and therefore may be utilized as a fundamental therapeutic agent for peripheral neuropathy.

Therefore, an object of the present invention is to provide a pharmaceutical composition for preventing or treating peripheral neuropathy, comprising an isoquinoline derivative represented by Chemical Formula 1 below or a pharmaceutically acceptable salt thereof as an active ingredient.

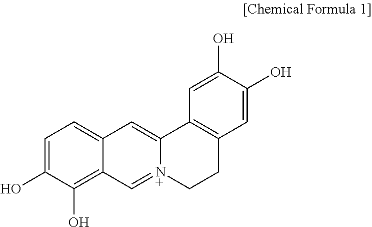 [SMILES: Oc1cc2c(cc1O)-c1cc3ccc(O)c(O)c3c[n+]1CC2]

Another object of the present invention is to provide a kit for preventing or treating peripheral neuropathy, comprising a composition including the isoquinoline derivative or a pharmaceutically acceptable salt thereof as an active ingredient, and instructions.

The present invention is also directed to providing a food composition for preventing or improving peripheral neuropathy, which includes an isoquinoline derivative or a sitologically acceptable salt thereof as an active ingredient.

However, technical problems to be solved in the present invention are not limited to the above-described problems, and other problems which are not described herein will be fully understood by those of ordinary skill in the art from the following descriptions.

### Technical Solution

The present invention provides a pharmaceutical composition for preventing or treating peripheral neuropathy, which includes an isoquinoline derivative represented by Chemical Formula 1 below or a pharmaceutically acceptable salt thereof as an active ingredient.

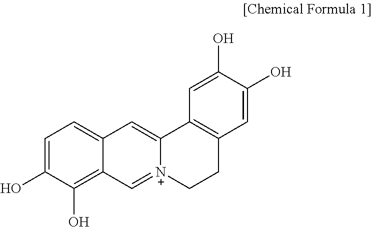 [SMILES: Oc1cc2c(cc1O)-c1cc3ccc(O)c(O)c3c[n+]1CC2]

The present invention also provides a method of preventing or treating peripheral neuropathy, which includes administering the isoquinoline derivative represented by Chemical Formula 1 or a pharmaceutically acceptable salt thereof to a subject in need thereof.

The present invention also provides a use of the isoquinoline derivative represented by Chemical Formula 1 or a pharmaceutically acceptable salt thereof to prevent or treat peripheral neuropathy.

The present invention also provides a use of the isoquinoline derivative represented by Chemical Formula 1 or a pharmaceutically acceptable salt thereof to prepare a drug for preventing or treating peripheral neuropathy.

The present invention also provides a kit for preventing or treating peripheral neuropathy, which includes the isoquinoline derivative represented by Chemical Formula 1, a pharmaceutically acceptable salt thereof, or the composition, and instructions.

The present invention also provides a food composition for preventing or improving peripheral neuropathy, which includes the isoquinoline derivative represented by Chemical Formula 1 or a sitologically acceptable salt thereof as an active ingredient.

In one embodiment of the present invention, the isoquinoline derivative or a pharmaceutically acceptable salt thereof may promote mitophagy activity, but the present invention is not limited thereto.

In another embodiment of the present invention, the isoquinoline derivative or a pharmaceutically acceptable salt thereof may satisfy, but is not limited to, one or more selected from the group consisting of:
(a) reducing the membrane potential of mitochondria;
(b) reducing the reactive oxygen species level in mitochondria; and
(c) increasing the ATP synthesis capacity of mitochondria.

In still another embodiment of the present invention, the isoquinoline derivative or a pharmaceutically acceptable salt thereof inhibit morphological degeneration of sensory nerves, but the present invention is not limited thereto.

In still another embodiment of the present invention, the isoquinoline derivative or a pharmaceutically acceptable salt thereof increase the number of peripheral nerves, but the present invention is not limited thereto.

In still another embodiment of the present invention, the peripheral neuropathy is chemotherapy-induced peripheral neuropathy, but the present invention is not limited thereto.

### Advantageous Effects

The present invention relates to a pharmaceutical composition for preventing or treating peripheral neuropathy, and the like. The present invention has been completed by confirming that an isoquinoline derivative discovered through a screening based on mitophage activity exhibits an excellent mitophage promoting effect, and thus, the isoquinoline derivative can be utilized as a fundamental therapeutic agent for peripheral neuropathy. Specifically, it has been found that the isoquinoline derivative according to the present invention treats hyperalgesia in an animal model of anticancer drug-inducible peripheral neuropathy, also suppresses morphological changes of sensory nerves, which have been identified as a key cause of peripheral neuropathy, and can increase the distribution and number of peripheral nerves. Therefore, the isoquinoline derivative according to the present invention is a fundamental therapeutic agent capable of suppressing the main symptoms and causes of peripheral neuropathy, particularly, anticancer drug-induced peripheral neuropathy, and is expected to be usefully utilized in the field of prevention, amelioration, and/or treatment of the disease.

### BRIEF DESCRIPTION OF THE DRAWINGS

FIGS. 1A to IC show the results of analyzing the promoting effect of mitophagy activity by the isoquinoline derivative compound according to an embodiment of the present invention (referred to as “CD1-012,” hereinafter the same) in a normal human lung cell line (FIG. 1A, FACS results; FIG. 1B, confocal microscopy observation results; and FIG. 1C, quantitative measurement of mitochondrial changes using mito-YFP fluorescent protein).

FIGS. 2A and 2B show the results of analyzing the promoting effect of mitophagy activity by CD1-012 in a SH-SY5Y cell line (FIG. 2A) and a Hela-Parkin cell line (FIG. 2B).

FIGS. 3A and 3B show the results of analyzing the mitophagy activity in BEAS-2B cells according to the treatment concentration (FIG. 3A) and treatment time (FIG. 3B) of CD1-012.

FIGS. 4A and 4B show the results of confirming changes in mitophagy activity (FIG. 4A) and autophagy activity (FIG. 4B) in cells treated with CD1-012.

FIG. 5 shows the result of comparing the promoting effect of mitophagy activity by CD1-012 compared with palmatine and berberine, according to concentration.

FIG. 6 shows the result of analyzing the levels of mitochondrial membrane potential and mitochondrial reactive oxygen species after treating cells with CD1-012 or the control compound CCCP.

FIG. 7 shows result of analyzing mitophagy promoting activity of CD1-012 and the control compound CCCP in PINK1 knockdown cell lines (shPINK1).

FIG. 8A shows the results of measuring the withdrawal latency of larvae in response to a heat probe after treatment of Drosophila larvae with an anticancer agent (paclitaxel) and/or CD1-012 to evaluate the therapeutic effect of CD1-012 on chemotherapy-induced peripheral neuropathy.

FIG. 8B shows the results of measuring larval size after treatment of Drosophila larvae with an anticancer agent and/or CD1-012 to evaluate the effect of CD1-012 on larval growth.

FIGS. 9A to 9C show the results of evaluating the inhibitory effect of CD1-012 on sensory neuron degeneration induced by an anticancer agent. Drosophila were treated with the anticancer agent and/or CD1-012, followed by observation of the morphology of C4da sensory neurons (FIG. 9A), and measurement of the total dendrite length (FIG. 9B) and the number of dendritic branches (FIG. 9C).

FIGS. 10A and 10B show the results of measuring the withdrawal latency of Drosophila larvae in response to a heat probe after treatment with an anticancer agent and/or CD1-012 in flies in which the expression of ATG5 (FIG. 10A) or ATG7 (FIG. 10B), which are mediators of the canonical mitophagy pathway, was suppressed.

FIGS. 11A and 11B show the results of measuring the withdrawal latency of Drosophila larvae in response to a heat probe after treatment with an anticancer agent and/or CD1-012 in flies in which the expression of ULK1 (FIG. 11A) or Rab9 (FIG. 11B), which are mediators of the alternative mitophagy pathway, was suppressed.

FIGS. 12A and 12B show the results of evaluating the therapeutic effect of CD1-012 on chemotherapy-induced peripheral neuropathy in mice. FIG. 12A illustrates the sensitivity to pain measured using the Von Frey Hair test after treatment with an anticancer agent (paclitaxel) and/or CD1-012. FIG. 12B shows the distribution of peripheral nerves in the footpad.

### BEST MODES OF THE INVENTION

The present invention relates to a pharmaceutical composition for preventing or treating peripheral neuropathy, and the like. The present invention has been completed by confirming that an isoquinoline derivative discovered through screening based on mitophagy activity exhibits an excellent mitophagy-promoting effect, and thus may be utilized as a fundamental therapeutic agent for peripheral neuropathy by improving major symptoms and blocking the pathogenesis thereof.

Specifically, the compound according to the present invention is an isoquinoline derivative with an identified mitophagy-specific promoting function and was confirmed to have no mitochondrial toxicity or cytotoxicity, and it has been confirmed through previous studies that it can improve mitochondrial dysfunction in an Alzheimer's disease model.

In addition, the inventors of the present invention confirmed the therapeutic effect of the compound on peripheral neuropathy by treating a Drosophila heat hyperalgesia model with the compound, and found that the heat hyperalgesia symptoms induced by the anticancer agent were alleviated to a normal level and that morphological changes in the sensory neurons were suppressed (Examples 5 and 6). In addition, it was confirmed in a mouse model of peripheral neuropathy that the compound improved heat hyperalgesia symptoms and increased the number of peripheral nerves in the footpad (Example 9).

Furthermore, the excellent therapeutic effect of the compound on peripheral neuropathy was found to be dependent on the alternative mitophagy pathway mediated by ULK1 and Rab9, while being independent of the canonical mitophagy pathway mediated by ATG5 and ATG7 (Example 7).

As described above, the novel isoquinoline derivative according to the present invention not only alleviates major symptoms of peripheral neuropathy but also effectively suppresses degeneration of sensory neurons and the reduction in peripheral nerve distribution, which are key causes of the disease. Accordingly, it is expected to be utilized as a fundamental therapeutic agent for peripheral neuropathy, particularly chemotherapy-induced peripheral neuropathy. In particular, since the mechanism of action of the compound in treating peripheral neuropathy has been identified through experiments at the molecular level, it is expected that peripheral neuropathy may be effectively treated in various patient populations through appropriate use of the compound based on its molecular mechanism.

Hereinafter, the present invention will be described in detail.

The present invention is directed to providing a pharmaceutical composition for preventing or treating peripheral neuropathy, which includes an isoquinoline derivative represented by Chemical Formula 1 below or a pharmaceutically acceptable salt thereof as an active ingredient.

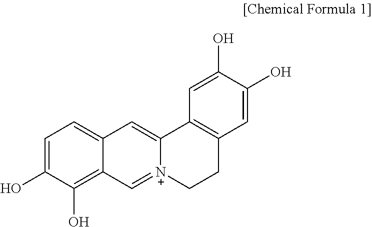 [SMILES: Oc1cc2c(cc1O)-c1cc3ccc(O)c(O)c3c[n+]1CC2]

In the present invention, the term “pharmaceutically acceptable salt” comprises a salt derived from pharmaceutically acceptable inorganic acids, organic acids, or bases.

The term “pharmaceutically acceptable” as used in the disclosure refers to a compound or composition that is suitable for use in contact with the tissues of a subject (e.g., a human) with a reasonable benefit/risk ratio, without excessive toxicity, irritation, allergic reaction, or other issues or complications, and that falls within the scope of sound medical judgment.

Examples of suitable acids include hydrochloric acid, bromic acid, sulfuric acid, nitric acid, perchloric acid, fumaric acid, maleic acid, phosphoric acid, glycolic acid, lactic acid, salicylic acid, succinic acid, toluene-p-sulfonic acid, tartaric acid, acetic acid, citric acid, methanesulfonic acid, formic acid, benzoic acid, malonic acid, gluconic acid, naphthalene-2-sulfonic acid, benzene sulfonic acid, etc. Acid addition salts may be manufactured by conventional methods, for example, by dissolving the compound in an excess of an acid aqueous solution and precipitating this salt using a water-miscible organic solvent such as methanol, ethanol, acetone, or acetonitrile. Acid addition salts may also be prepared by heating an equimolar amount of the compound and an acid or alcohol in water and subsequently evaporating the mixture to dryness, or by aspiration-filtration of the precipitated salt.

Salts derived from suitable bases may include alkali metals such as sodium, potassium, alkaline earth metals such as magnesium, and ammonium, but are not limited thereto. Alkali metal or alkaline earth metal salts, for example, may be obtained by dissolving the compound in an excess of alkali metal hydroxide or alkaline earth metal hydroxide solution, filtering off the insoluble compound salt, and then evaporating and drying the filtrate. Here, it is pharmaceutically suitable to manufacture particularly sodium, potassium, or calcium salts as the metal salts, and the corresponding silver salts may be obtained by reacting an alkali metal or alkaline earth metal salt with a suitable silver salt (for example, silver nitrate).

The scope of the compounds of the present invention includes not only pharmaceutically acceptable salts but also all isomers, hydrates, and solvates that can be prepared by conventional methods.

In one embodiment of the present invention, the isoquinoline derivative represented by Chemical Formula 1 may be prepared as shown in Reaction Scheme 1 below according to a preparation method that includes adding palmatine represented by Chemical Formula 2 or berberine represented by Chemical Formula 3 and a Lewis acid catalyst to an organic solvent to allow a reaction (Step 1).

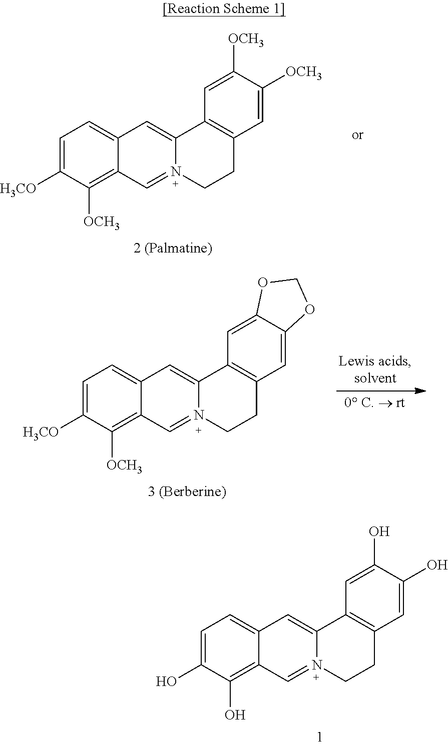

According to an embodiment of the present invention, the isoquinoline derivative or a pharmaceutically acceptable salt thereof may be a derivative in which a hydrophobic substituent (methoxy group) in the core structure of the palmatine or berberine is substituted with a hydrophilic substituent or a functional group that can provide a hydrogen bond between molecules (hydroxyl group). For example, the isoquinoline derivative according to the present invention may be selected from 2,3,5,10-tetrahydroxy-5,6-dihydroisoquinolino[3,2-a]isoquinolin-7-ium bromide (2,3,9,10-tetrahydroxy-5,6-dihydroisoquinolino[3,2-a]isoquinolin-7-ium bromide), 2,3,5,10-tetrahydroxy-5,6-dihydroisoquinolino[3,2-a]isoquinolin-7-ium hydroxide (2,3,9,10-tetrahydroxy-5,6-dihydroisoquinolino[3,2-a]isoquinolin-7-ium hydroxide), and 2,3,5,10-tetrahydroxy-5,6-dihydroisoquinolino[3,2-a]isoquinolin-7-ium chloride (2,3,9,10-tetrahydroxy-5,6-dihydroisoquinolino[3,2-a]isoquinolin-7-ium chloride), which are represented by Chemical Formulas 1a to 1c, respectively. In the disclosure, the 2,3,5,10-yetrahydroxy-5,6-dihydroisoquinolino[3,2-a]isoquinolin-7-ium bromide may be referred to as “CD1-012.”

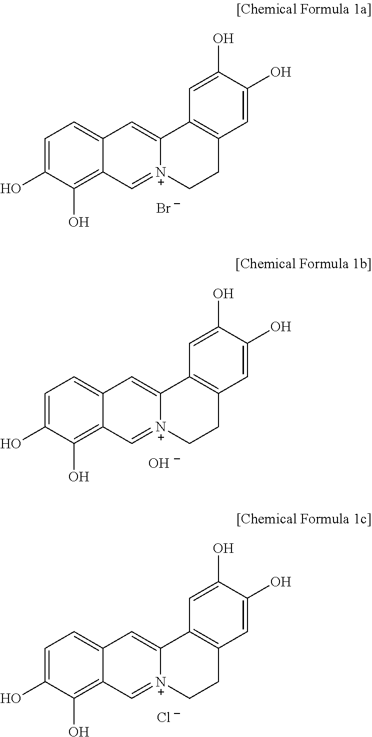

The isoquinoline derivative according to the present invention or a pharmaceutically acceptable salt thereof is characterized by promoting mitophagy activity (i.e., activating mitophagy).

In the present invention, the term “mitophagy” refers to an intracellular degradation mechanism which removes damaged or unnecessary mitochondria. Mitophagy is a process in which autophagosomes are formed when mitochondria are damaged, and fused with lysosomes to selectively degrade and remove the damaged mitochondria. Mitophagy is a distinct mechanism from autophagy, which is a mechanism that decomposes and recycles unnecessary components within cells (old proteins, protein aggregates, organelles, pathogens that have invaded cells, etc.) to produce a macromolecular precursor and energy when the cells are under nutrient deficiency. Mitophagy is regulated independently of regulatory signals such as nutrients, energy, and stress, which control autophagy. The mitophagy is classified into canonical mitophagy and alternative mitophagy (also referred to as non-canonical mitophagy). Canonical mitophagy involves ATG proteins such as ATG5 and ATG7, whereas alternative mitophagy is independent of ATG proteins and is mediated by Ulk1/Rab9/Rip1.

The inventors confirmed through specific examples that the isoquinoline derivative according to the present invention exhibits a superior mitophagy-activating effect compared to other known mitophagy activators. Based on this, it was also confirmed that the compound improves major symptoms of chemotherapy-induced peripheral neuropathy and suppresses key pathologies such as degeneration of sensory neurons and reduction in peripheral nerves. Therefore, the isoquinoline derivative according to the present invention or a pharmaceutically acceptable salt thereof may exhibit an excellent therapeutic effect particularly in a patient with peripheral neuropathy having a decreased mitophagy level (e.g., a mitophagy level of neurons).

In particular, specific examples demonstrated that the mechanism by which the compound of the present invention treats peripheral neuropathy is independent of ATG5 or ATG7, indicating that the compound can treat peripheral neuropathy independently of the canonical mitophagy pathway. That is, the compound of the present invention may exhibit excellent therapeutic effects even in patients with peripheral neuropathy in whom the canonical mitophagy pathway is inactivated due to mutations in the canonical mitophagy pathway or other causes. The mutation in the canonical mitophagy pathway includes inactivation or reduced activity of the canonical mitophagy pathway, which may be caused by a decrease in the level or activity of proteins involved in the canonical mitophagy pathway (e.g., ATG proteins such as ATG5, ATG7, and ATG8).

In addition, in an embodiment of the present invention, the therapeutic mechanism of the compound for peripheral neuropathy may be dependent on the alternative mitophagy pathway. That is, the compound may exert a therapeutic effect on peripheral neuropathy by promoting mitophagy through the alternative mitophagy pathway. Therefore, an excellent therapeutic effect on peripheral neuropathy may be achieved by activating the alternative mitophagy pathway in conjunction with the administration of the compound, but the present invention is not limited thereto. Examples of activation of the alternative mitophagy pathway include, but are not limited to, overexpression or activation of mediators of the alternative mitophagy pathway, such as ULK1 and Rab9.

In addition, the isoquinoline derivative according to the present invention or a pharmaceutically acceptable salt thereof may satisfy one or more properties selected from the group consisting of:
(a) reducing the membrane potential of mitochondria;
(b) reducing the reactive oxygen species (ROS) level in mitochondria; and
(c) increasing he ATP synthesis capacity of mitochondria.

That is, the isoquinoline derivative according to the present invention or a pharmaceutically acceptable salt thereof may raise the level and/or activity (function) of mitochondria. The effect of the compound according to the present invention on improving the mitochondrial level and/or activity may be achieved by the mitophagy activating effect of the compound. Accordingly, the isoquinoline derivative according to the present invention or a pharmaceutically acceptable salt thereof may exhibit an excellent therapeutic effect particularly in a patient with peripheral neuropathy having the decreased mitochondrial level and/or function (e.g., the level and/or function of mitochondria in neurons).

In addition, in the present invention, the isoquinoline derivative or a pharmaceutically acceptable salt thereof may be characterized by inhibiting (alleviating or improving) morphological degeneration of sensory neurons. The hyperalgesia observed in patients with peripheral neuropathy is known to be primarily caused by morphological changes in sensory neurons. For example, in chemotherapy-induced peripheral neuropathy, chemotherapeutic agents induce an increase in the length and number of dendritic arbors of sensory neurons, which may lead to hyperalgesia. Therefore, the compound of the present invention may inhibit an increase in the length of dendritic arbors of sensory neurons or may inhibit an increase in the number of branches (i.e., the number of ramification). Preferably, the sensory neuron may be a Class IV da (C4da) sensory neuron. Accordingly, the compound of the present invention may exhibit an especially excellent therapeutic effect in patients with peripheral neuropathy accompanied by morphological degeneration of sensory neurons (an increase in the length or number of dendritic arbors).

The isoquinoline derivative or a pharmaceutically acceptable salt thereof may increase the number of peripheral nerves. Peripheral neuropathy is caused by dysfunction of peripheral nerves and a reduction in normal peripheral nerves, and it has been confirmed through specific examples that the compound of the present invention can increase the number and distribution of peripheral nerves. Accordingly, the compound of the present invention may exhibit an excellent therapeutic effect particularly in a patient with peripheral neuropathy having a reduced number or distribution of peripheral nerves. The peripheral nerves are preferably intraepidermal nerve fibers (IENFs).

In the present invention, “peripheral neuropathy” refers to a neurological disorder in which various problems in bodily functions occur due to damage to or dysfunction of the peripheral nervous system. Patients with peripheral neuropathy may experience sensory abnormalities such as tingling in the feet, burning sensations, numbness, and severe pain (hyperalgesia), as well as loss of balance or muscle strength. Peripheral neuropathy is induced by various causes that directly or indirectly affect nerve tissues. For example, acute peripheral neuropathy may be caused by infections, autoimmune responses, drugs, or toxic substances such as chemotherapeutic agents, while chronic peripheral neuropathy is induced by metabolic disorders such as diabetes, alcoholism, nutritional deficiency, renal failure, or hepatic failure. In one embodiment of the present invention, the peripheral neuropathy may be “chemotherapy-induced peripheral neuropathy (CIPN)” caused by a chemotherapeutic agent. This is a representative form of acquired peripheral neuropathy, occurring in more than 60% of patients undergoing chemotherapy. It is mostly induced in a dose-dependent manner according to the cumulative dose of the chemotherapeutic agent and causes severe pain such as sensory abnormalities, allodynia (pain from non-painful stimuli), and hyperalgesia. The chemotherapeutic agent is not particularly limited as long as it can cause dysfunction or damage to peripheral nerves, and includes platinum-based agents, taxane-based agents, vinca alkaloids, proteasome inhibitors, bortezomib, and thalidomide, as well as all other chemotherapeutic agents that are clinically, pharmaceutically, or biomedically applicable. Specifically, the chemotherapeutic agent may be selected from the group consisting of paclitaxel, bortezomib, oxaliplatin, vincristine, cisplatin, Taxol, docetaxel, ixabepilone, thalidomide, Velcade, and lenalidomide. The compound of the present invention may be used for alleviating, improving, preventing, and/or treating chemotherapy-induced peripheral neuropathic pain caused by the chemotherapeutic agent.

The present invention provides a pharmaceutical composition for preventing or treating peripheral neuropathy, comprising an isoquinoline derivative represented by Chemical Formula 1 below or a pharmaceutically acceptable salt thereof as an active ingredient.

In one embodiment of the present invention, the isoquinoline derivative or a pharmaceutically acceptable salt thereof specifically promotes mitophagy activity, but is not limited thereto.

In one embodiment of the present invention, the mitophagy activity may be independent of one or more selected from the group consisting of PINK1, ATG5, and ATG7, but is not limited thereto.

In one embodiment of the present invention, the mitophagy activity may be dependent on ULK1 or Rap9, but is not limited thereto.

In one embodiment of the present invention, the isoquinoline derivative or a pharmaceutically acceptable salt thereof may inhibit morphological degeneration of sensory neurons, but is not limited thereto.

In one embodiment of the present invention, the isoquinoline derivative or a pharmaceutically acceptable salt thereof may increase the number of peripheral nerves, but is not limited thereto.

In one embodiment of the present invention, the peripheral neuropathy may be chemotherapy-induced peripheral neuropathy, but is not limited thereto.

In one embodiment of the present invention, the chemotherapeutic agent may be one selected from the group consisting of paclitaxel, bortezomib, oxaliplatin, vincristine, cisplatin, taxol, docetaxel, ixabepilone, thalidomide, Velcade, and lenalidomide, but is not limited thereto.

In one embodiment of the present invention, the isoquinoline derivative or a pharmaceutically acceptable salt thereof may be present in an amount of greater than 0 and up to 40 μM, but is not limited thereto.

In one embodiment of the present invention, the isoquinoline derivative or a pharmaceutically acceptable salt thereof may improve mitochondrial dysfunction, but is not limited thereto.

In one embodiment of the present invention, the improvement in mitochondrial dysfunction may be one selected from the group consisting of:
(a) maintaining the normal membrane potential level in mitochondria;
(b) reducing the reactive oxygen species (ROS) level in mitochondria; and
(c) increasing the ATP synthesis capacity of mitochondria,
but is not limited thereto.

The present invention provides a kit for preventing or treating peripheral neuropathy, comprising the pharmaceutical composition and instructions.

The present invention provides a food composition for preventing or improving peripheral neuropathy, comprising an isoquinoline derivative represented by Chemical Formula 1 or a sitologically acceptable salt thereof as an active ingredient.

In one embodiment of the present invention, the peripheral neuropathy may be chemotherapy-induced peripheral neuropathy, but is not limited thereto. The content of the isoquinoline derivative or pharmaceutically acceptable salt thereof in the composition of the present invention may be appropriately adjusted depending on the symptoms of a disease, the degree of progression of symptoms, the condition of a patient, and the like, and may range from, for example, 0.0001 wt % to 99.9 wt % or 0.001 wt % to 50 wt % with respect to a total weight of the composition, but the present invention is not limited thereto. The amount ratio is a value based on the amount of dried product from which a solvent is removed.

The pharmaceutical composition according to the present invention may further include a suitable carrier, excipient, and diluent which are commonly used in the preparation of pharmaceutical compositions. The excipient may be, for example, one or more selected from the group consisting of a diluent, a binder, a disintegrant, a lubricant, an adsorbent, a humectant, a film-coating material, and a controlled release additive.

The pharmaceutical composition according to the present invention may be used by being formulated, according to commonly used methods, into a form such as powders, granules, sustained-release-type granules, enteric granules, liquids, eye drops, elixirs, emulsions, suspensions, spirits, troches, aromatic water, lemonades, tablets, sustained-release-type tablets, enteric tablets, sublingual tablets, hard capsules, soft capsules, sustained-release-type capsules, enteric capsules, pills, tinctures, soft extracts, dry extracts, fluid extracts, injections, capsules, perfusates, or a preparation for external use, such as plasters, lotions, pastes, sprays, inhalants, patches, sterile injectable solutions, or aerosols. The preparation for external use may have a formulation such as creams, gels, patches, sprays, ointments, plasters, lotions, liniments, pastes, or cataplasmas.

As the carrier, the excipient, and the diluent that may be included in the pharmaceutical composition according to the present invention, lactose, dextrose, sucrose, oligosaccharides, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil may be used.

For formulation, commonly used diluents or excipients such as fillers, thickeners, binders, wetting agents, disintegrants, and surfactants are used.

As additives of tablets, powders, granules, capsules, pills, and troches according to the present invention, excipients such as corn starch, potato starch, wheat starch, lactose, white sugar, glucose, fructose, D-mannitol, precipitated calcium carbonate, synthetic aluminum silicate, dibasic calcium phosphate, calcium sulfate, sodium chloride, sodium hydrogen carbonate, purified lanolin, microcrystalline cellulose, dextrin, sodium alginate, methyl cellulose, sodium carboxymethylcellulose, kaolin, urea, colloidal silica gel, hydroxypropyl starch, hydroxypropyl methylcellulose (HPMC), HPMC 1928, HPMC 2208, HPMC 2906, HPMC 2910, propylene glycol, casein, calcium lactate, and Primojel®; and binders such as gelatin, Arabic gum, ethanol, agar powder, cellulose acetate phthalate, carboxymethylcellulose, calcium carboxymethylcellulose, glucose, purified water, sodium caseinate, glycerin, stearic acid, sodium carboxymethylcellulose, sodium methylcellulose, methylcellulose, microcrystalline cellulose, dextrin, hydroxycellulose, hydroxypropyl starch, hydroxymethylcellulose, purified shellac, starch, hydroxypropyl cellulose, hydroxypropyl methylcellulose, polyvinyl alcohol, and polyvinylpyrrolidone may be used, and disintegrants such as hydroxypropyl methylcellulose, corn starch, agar powder, methylcellulose, bentonite, hydroxypropyl starch, sodium carboxymethylcellulose, sodium alginate, calcium carboxymethylcellulose, calcium citrate, sodium lauryl sulfate, silicic anhydride, 1-hydroxypropylcellulose, dextran, ion-exchange resin, polyvinyl acetate, formaldehyde-treated casein and gelatin, alginic acid, amylose, guar gum, sodium bicarbonate, polyvinylpyrrolidone, calcium phosphate, gelled starch, Arabic gum, amylopectin, pectin, sodium polyphosphate, ethyl cellulose, white sugar, magnesium aluminum silicate, a di-sorbitol solution, and light anhydrous silicic acid; and lubricants such as calcium stearate, magnesium stearate, stearic acid, hydrogenated vegetable oil, talc, lycopodium powder, kaolin, Vaseline, sodium stearate, cacao butter, sodium salicylate, magnesium salicylate, polyethylene glycol (PEG) 4000, PEG 6000, liquid paraffin, hydrogenated soybean oil (Lubri wax), aluminum stearate, zinc stearate, sodium lauryl sulfate, magnesium oxide, Macrogol, synthetic aluminum silicate, silicic anhydride, higher fatty acids, higher alcohols, silicone oil, paraffin oil, polyethylene glycol fatty acid ether, starch, sodium chloride, sodium acetate, sodium oleate, dl-leucine, and light anhydrous silicic acid may be used.

As additives of liquids according to the present invention, water, dilute hydrochloric acid, dilute sulfuric acid, sodium citrate, monostearic acid sucrose, polyoxyethylene sorbitol fatty acid esters (twin esters), polyoxyethylene monoalkyl ethers, lanolin ethers, lanolin esters, acetic acid, hydrochloric acid, ammonia water, ammonium carbonate, potassium hydroxide, sodium hydroxide, prolamine, polyvinylpyrrolidone, ethylcellulose, and sodium carboxymethylcellulose may be used.

In syrups according to the present invention, a white sugar solution, other sugars or sweeteners, and the like may be used, and as necessary, a fragrance, a colorant, a preservative, a stabilizer, a suspending agent, an emulsifier, a viscous agent, or the like may be used.

In emulsions according to the present invention, purified water may be used, and as necessary, an emulsifier, a preservative, a stabilizer, a fragrance, or the like may be used.

In suspensions according to the present invention, suspending agents such as acacia, tragacanth, methylcellulose, carboxymethylcellulose, sodium carboxymethylcellulose, microcrystalline cellulose, sodium alginate, hydroxypropyl methylcellulose (HPMC), HPMC 1828, HPMC 2906, HPMC 2910, and the like may be used, and as necessary, a surfactant, a preservative, a stabilizer, a colorant, and a fragrance may be used.

Injections according to the present invention may include: solvents such as distilled water for injection, a 0.9% sodium chloride solution, Ringer's solution, a dextrose solution, a dextrose+sodium chloride solution, PEG, lactated Ringer's solution, ethanol, propylene glycol, non-volatile oil-sesame oil, cottonseed oil, peanut oil, soy bean oil, corn oil, ethyl oleate, isopropyl myristate, and benzene benzoate; cosolvents such as sodium benzoate, sodium salicylate, sodium acetate, urea, urethane, monoethylacetamide, butazolidine, propylene glycol, the Tween series, amide nicotinate, hexamine, and dimethylacetamide; buffers such as weak acids and salts thereof (acetic acid and sodium acetate), weak bases and salts thereof (ammonia and ammonium acetate), organic compounds, proteins, albumin, peptone, and gums; isotonic agents such as sodium chloride; stabilizers such as sodium bisulfite (NaHSO₃) carbon dioxide gas, sodium metabisulfite (Na₂S₂O₅), sodium sulfite (Na₂SO₃), nitrogen gas (N₂), and ethylenediamine tetraacetic acid; sulfating agents such as 0.1% sodium bisulfide, sodium formaldehyde sulfoxylate, thiourea, disodium ethylenediaminetetraacetate, and acetone sodium bisulfite; a pain relief agent such as benzyl alcohol, chlorobutanol, procaine hydrochloride, glucose, and calcium gluconate; and suspending agents such as sodium CMC, sodium alginate, Tween 80, and aluminum monostearate.

In suppositories according to the present invention, bases such as cacao butter, lanolin, Witepsol, polyethylene glycol, glycerogelatin, methylcellulose, carboxymethylcellulose, a mixture of stearic acid and oleic acid, Subanal, cottonseed oil, peanut oil, palm oil, cacao butter+cholesterol, lecithin, lanette wax, glycerol monostearate, Tween or span, imhausen, monolan (propylene glycol monostearate), glycerin, Adeps solidus, buytyrum Tego-G, cebes Pharma 16, hexalide base 95, cotomar, Hydrokote SP, S-70-XXA, S-70-XX75 (S-70-XX95), Hydrokote 25, Hydrokote 711, idropostal, massa estrarium (A, AS, B, C, D, E, I, T), masa-MF, masupol, masupol-15, neosuppostal-N, paramount-B, supposiro OSI, OSIX, A, B, C, D, H, L, suppository base IV types (AB, B, A, BC, BBG, E, BGF, C, D, 299), suppostal (N, Es), Wecoby (W, R, S, M, Fs), and tegester triglyceride matter (TG-95, MA, 57) may be used.

Solid preparations for oral administration include tablets, pills, powders, granules, capsules, and the like, and such solid preparations are formulated by mixing the composition with at least one excipient, e.g., starch, calcium carbonate, sucrose, lactose, gelatin, and the like. In addition to simple excipients, lubricants such as magnesium stearate and talc are also used.

Examples of liquid preparations for oral administration include suspensions, liquids for internal use, emulsions, syrups, and the like, and these liquid preparations may include, in addition to simple commonly used diluents, such as water and liquid paraffin, various types of excipients, for example, a wetting agent, a sweetener, a fragrance, a preservative, and the like. Preparations for parenteral administration include an aqueous sterile solution, a non-aqueous solvent, a suspension, an emulsion, a freeze-dried preparation, and a suppository. Non-limiting examples of the non-aqueous solvent and the suspension include propylene glycol, polyethylene glycol, a vegetable oil such as olive oil, and an injectable ester such as ethyl oleate.

The pharmaceutical composition according to the present invention is administered in a pharmaceutically effective amount. In the present invention, “the pharmaceutically effective amount” refers to an amount sufficient to treat diseases at a reasonable benefit/risk ratio applicable to medical treatment, and an effective dosage level may be determined according to factors including types of diseases of patients, the severity of disease, the activity of drugs, sensitivity to drugs, administration time, administration route, excretion rate, treatment period, and simultaneously used drugs, and factors well known in other medical fields.

The composition according to the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects, in consideration of all the aforementioned factors, and this may be easily determined by those of ordinary skill in the art.

The pharmaceutical composition of the present invention may be administered to a subject via various routes. All administration methods can be predicted, and the pharmaceutical composition may be administered via, for example, oral administration, subcutaneous injection, intraperitoneal injection, intravenous injection, intramuscular injection, intrathecal (space around the spinal cord) injection, sublingual administration, administration via the buccal mucosa, intrarectal insertion, intravaginal insertion, ocular administration, intra-aural administration, intranasal administration, inhalation, spraying via the mouth or nose, transdermal administration, percutaneous administration, or the like.

The pharmaceutical composition of the present invention is determined by the type of drug that is the active ingredient, along with other relevant factors such as the disease to be treated, the route of administration, and the patient's age, gender, weight, and severity of the disease. Specifically, the effective amount of the composition according to the present invention may vary depending on the age, gender, and weight of the patient. Generally, it may be administered daily or every other day at a dose of 0.001 to 150 mg per 1 kg of body weight, preferably 0.01 to 100 mg per 1 kg of body weight, or divided into one to three doses per day. However, the dosage may be increased or decreased depending on the route of administration, severity of the disease, gender, weight, and age, and therefore, the above dosage range should not be construed as limiting the scope of the present invention in any way.

As used herein, the “subject” refers to a subject in need of treatment of a disease, and more specifically, refers to a mammal such as a human or a non-human primate, a mouse, a rat, a dog, a cat, a horse, and a cow, but the present invention is not limited thereto.

As used herein, the “administration” refers to providing a subject with a predetermined composition of the present invention by using an arbitrary appropriate method.

The term “prevention” as used herein means all actions that inhibit or delay the onset of a target disease. The term “treatment” as used herein means all actions that alleviate or beneficially change a target disease and abnormal metabolic symptoms caused thereby via administration of the pharmaceutical composition according to the present invention. The term “improvement” as used herein means all actions that reduce the degree of parameters related to a target disease, e.g., symptoms via administration of the composition according to the present invention.

In addition, the present invention provides a kit for preventing or treating peripheral neuropathy, which includes the isoquinoline derivative according to the present invention or a pharmaceutically acceptable salt thereof.

In the present invention, the term “kit” refers to a tool used for the prevention or treatment of peripheral neuropathy, which includes the isoquinoline derivative of the present invention, a pharmaceutically acceptable salt thereof, or a composition including the same. The kit may further include other components, compositions, solutions, or devices that are conventionally required for the preparation, storage, or administration of the above substances, in addition to the compound or composition. For example, the kit may include an instruction manual describing the characteristics of the isoquinoline derivative according to the present invention or pharmaceutically acceptable salt thereof, as well as guidelines on proper use and storage.

Furthermore, the present invention provides a method for preparing the isoquinoline derivative or a pharmaceutically acceptable salt thereof, including a step of reacting palmatine or berberine with a Lewis acid catalyst in an organic solvent.

Specifically, the preparation method includes a step of reacting palmatine, represented by Chemical Formula 2, or berberine, represented by Chemical Formula 3, with a Lewis acid catalyst in an organic solvent, which can be represented by Reaction Scheme 1 below.

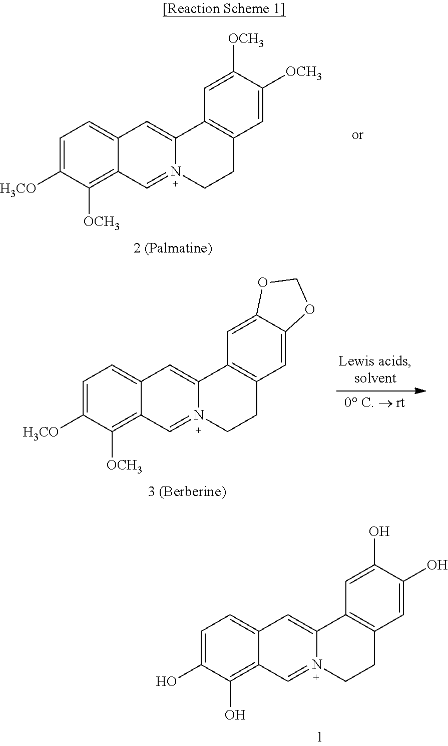

In one embodiment of the present invention, the Lewis acid catalyst may be at least one of metal halides such as BF₃, BBr₃, AlF₃, AlCl₃, AlBr₃, TiCl₄, TiBr₄, TiI₄, FeCl₃, FeCl₂, SnCl₂, SnCl₄, WCl₆, MoCl₅, SbCl₅, TeCl₂, and ZnCl₂; metal alkyl compounds such as Et₃Al, Et₂AlCl, EtAlCl₂, Et₃Al₂Cl₃, (i-Bu)₃Al, (i-Bu)₂AlCl, (i-Bu) AlCl₂, Me₄Sn, Et₄Sn, Bu₄Sn, and Bu₃SnCl; and metal alkoxy compounds such as Al(OR)₃₋ₓClₓ or Ti(OR)_{4-y}Cl_{y} (wherein the R represents an alkyl or aryl group, x is 1 or 2, y is an integer of 1 to 3), for example, a metal halide, for example, BBr₃, but is not limited thereto.

In one embodiment of the present invention, the organic solvent may be any one and more selected from the group consisting of dimethyl sulfoxide, dimethyl formamide, acetone, tetrahydrofuran, benzene, toluene, ether, methanol, hexane, cyclohexane, pyridine, acetic acid, carbon tetrachloride, chloroform, dichloromethane, and water, for example, dichloromethane, but is not limited thereto.

In one embodiment of the present invention, the addition of the Lewis acid catalyst may be performed in an inert gas. Specifically, the Lewis acid catalyst may be performed by using a method of adding to the palmatine or berberine dissolved in the organic solvent at about 0° C., in an inert gas atmosphere, for example, under a nitrogen stream.

In one embodiment of the present invention, after adding the Lewis acid catalyst, the reaction can be stirred for 10 to 14 hours, for example, 11 to 13 hours, for example, 12 hours, at room temperature, for example, 20° C. to 28° C., for example, 24° C. to 26° C., and the completion of the reaction may be confirmed, for example, using TLC (thin-layer Chromatography), but is not limited thereto.

Furthermore, the present invention provides a food composition for preventing or improving peripheral neuropathy, which includes an isoquinoline derivative or a sitologically acceptable salt thereof. The food composition includes a health functional food composition.

In the present invention, the term “sitologically acceptable salt” comprises a salt derived from pharmaceutically acceptable inorganic acids, organic acids, or bases.

The isoquinoline derivative of present invention or a sitologically acceptable salt thereof may be used by adding the compound as is to food or may be used together with other foods or food ingredients, but may be appropriately used according to a typical method. The mixed amount of the active ingredient may be suitably determined depending on the purpose of use thereof (for prevention, health, or therapeutic treatment). In general, when a food or beverage is prepared, the compound of the present invention is added in an amount of 15 wt % or less, preferably 10 wt % or less based on the raw materials. However, for long-term intake for the purpose of health and hygiene or for the purpose of health control, the amount may be less than the above-mentioned range, and the vesicles have no problem in terms of stability, so the active ingredient may be used in an amount more than the above-mentioned range.

The type of food is not particularly limited. Examples of food to which the material may be added include meats, sausage, bread, chocolate, candies, snacks, confectioneries, pizza, instant noodles, other noodles, gums, dairy products including ice creams, various soups, beverages, tea, drinks, alcoholic beverages, vitamin complexes, and the like, and include all health functional foods in a typical sense.

The health beverage composition according to the present invention may contain various flavors or natural carbohydrates, and the like as additional ingredients as in a typical beverage. The above-described natural carbohydrates may be monosaccharides such as glucose and fructose, disaccharides such as maltose and sucrose, polysaccharides such as dextrin and cyclodextrin, and sugar alcohols such as xylitol, sorbitol, and erythritol. As a sweetener, it is possible to use a natural sweetener such as thaumatin and stevia extract, a synthetic sweetener such as saccharin and aspartame, and the like. The proportion of the natural carbohydrates is generally about 0.01 to 0.20 g, or about 0.04 to 0.10 g per 100 ml of the composition of the present invention.

In addition to the aforementioned ingredients, the composition of the present invention may contain various nutrients, vitamins, electrolytes, flavors, colorants, pectic acids and salts thereof, alginic acid and salts thereof, organic acids, protective colloid thickeners, pH adjusters, stabilizers, preservatives, glycerin, alcohols, carbonating agents used in carbonated drinks, and the like. In addition, the composition of the present invention may contain flesh for preparing natural fruit juice, fruit juice drinks, and vegetable drinks. These ingredients may be used either alone or in combinations thereof. The proportion of these additives is not significantly important, but is generally selected within a range of 0.01 to 0.20 part by weight per 100 parts by weight of the composition of the present invention.

In this specification, the term “health functional food” is synonymous with “food for special health use (FoSHU)” and refers to foods that have been processed to efficiently exhibit bio-regulatory functions in addition to providing nutrition, demonstrating high medical and healthcare effects. Such foods may be manufactured in various forms, including tablets, capsules, powders, granules, liquids, and pills, to achieve beneficial effects in the prevention or improvement of peripheral neuropathy.

The health functional food of the present invention can be prepared using conventional methods known in the field, and during its preparation, commonly used raw materials and ingredients in the industry may be added. Additionally, unlike general pharmaceuticals, health functional foods are made from food-based ingredients, offering the advantage of avoiding side effects that may occur with long-term medication use, while also being highly portable.

### MODES OF THE INVENTION

Hereinafter, preferable examples are presented to aid in the understanding of the present invention. However, these examples are provided merely for easier understanding of the present invention, and the content of the present invention is not limited by these examples.

### EXAMPLE

### Example 1. Synthesis of Isoquinoline Derivative Compound CD1-012

The isoquinoline derivative compound 2,3,5,10-Tetrahydroxy-5,6-dihydroisoquinolino[3,2-a]isoquinolin-7-iumbromide (2,3,9,10-Tetrahydroxy-5,6-dihydroisoquinolino[3,2-a]isoquinolin-7-iumbromide; CD1-012) according to the present invention was synthesized through the following process: In a dried 250 mL round flask, palmatine (1.0 g, 2.92 mmol) was dissolved in 40 mL of anhydrous CH₂Cl₂. A BBr₃ solution (12.80 mL, 12.80 mmol) was added under a nitrogen atmosphere at 0° C. to prepare the reaction mixture. The reaction mixture was stirred at room temperature for 12 hours, and the completion of the reaction was confirmed using TLC. After the completion of the reaction, 10 mL of MeOH was added to the reaction mixture and stirred for 30 minutes. It was then concentrated via a vacuum concentrator, washed with CH₂Cl₂ (100 mL×5), and dried under reduced pressure to yield a solid form of the isoquinoline derivative compound (2,3,9,10-Tetrahydroxy-5,6-dihydroisoquinolino[3,2-a]isoquinolin-7-iumbromide; CD1-012) with a yield of 99% (1.09 g, 2.98 mmol). The reaction is represented by Reaction Formula 1a below:

¹H-NMR (400 MHz, DMSO-d₆) δ10.71 (s, 1H), 10.62 (s, 1H) 9.98 (br, 1H), 9.74 (s, 1H), 9.23 (br, 1H), 8.58 (s, 1H), 7.75 (d, J=8.8 Hz, 1H) 7.61 (d, J=8.8 Hz 1H), 7.46 (s, 1H), 6.77 (s, 1H), 4.82 (t, J=6.0 Hz, 2H), 3.06 (t, J=6.0 Hz, 2H)

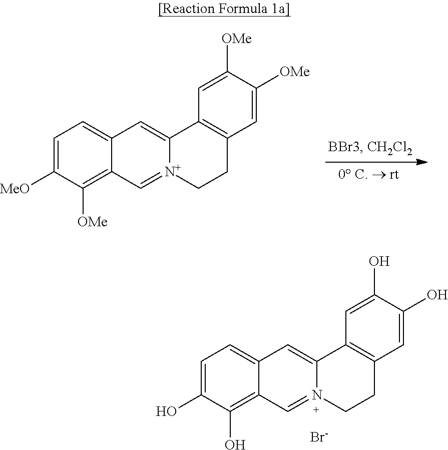

### Comparative Example 1. Carbonyl cyanide 3-chlorophenylhydrazone (CCCP)

CCCP, a representative compound promoting mitophagy, was used as Comparative Example 1.

### Comparative Example 2. Palmatine

Palmatine (CAS Number: 3486-67-7), represented by Chemical Formula 2 below, was used as Comparative Example 2.

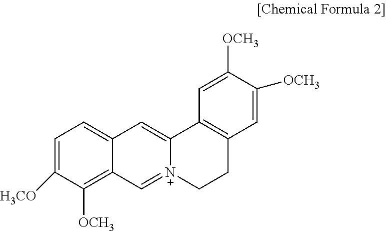 [SMILES: COc1cc2c(cc1OC)-c1cc3ccc(OC)c(OC)c3c[n+]1CC2]

### Comparative Example 3. Berberine

Berberine, represented by Chemical Formula 3 below and with CAS Numbers: 633-65-8 (Berberine·HCl) or 2086-83-1 (Berberine·HCl·2H₂O), was used as Comparative Example 3.

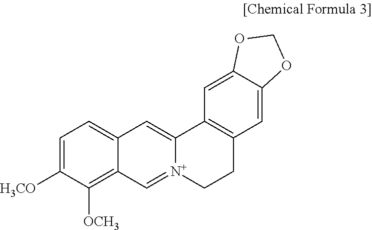 [SMILES: COc1ccc2cc3[n+](cc2c1OC)CCc1cc2c(cc1-3)OCO2]

### Example 2. Analysis of Mitophagy Activity Promoting Effect of CD1-012

### Example 2-1. Analysis of Mitophagy Activity Promoting Effect

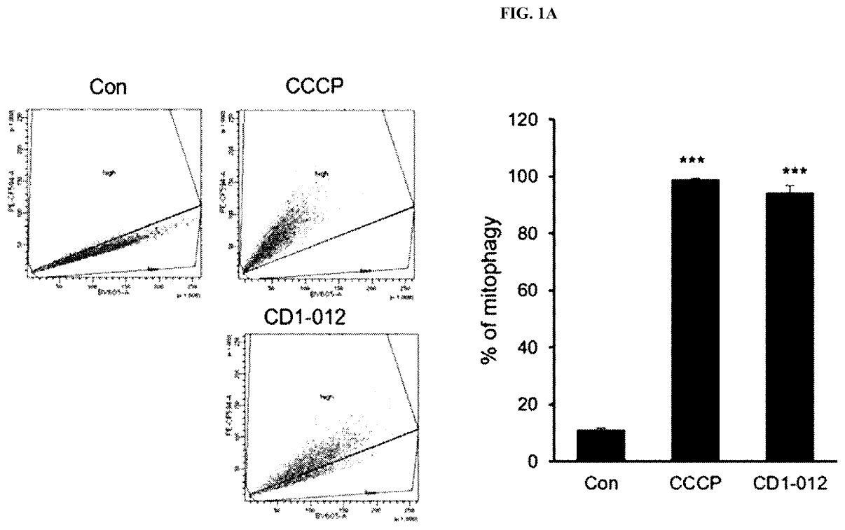

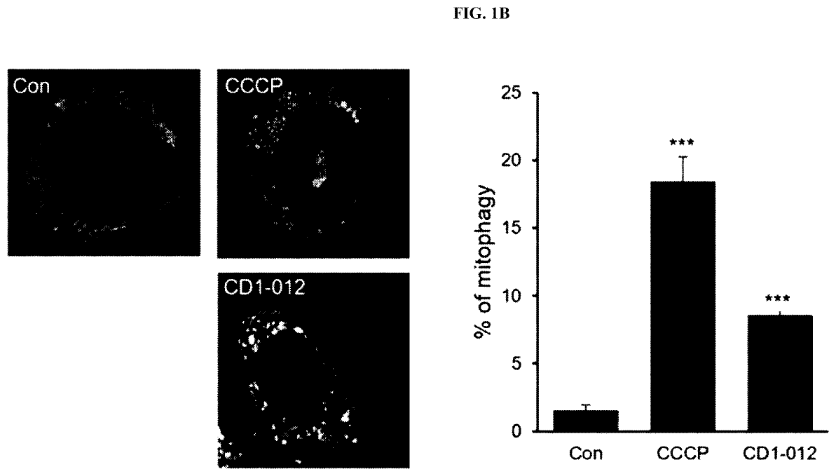

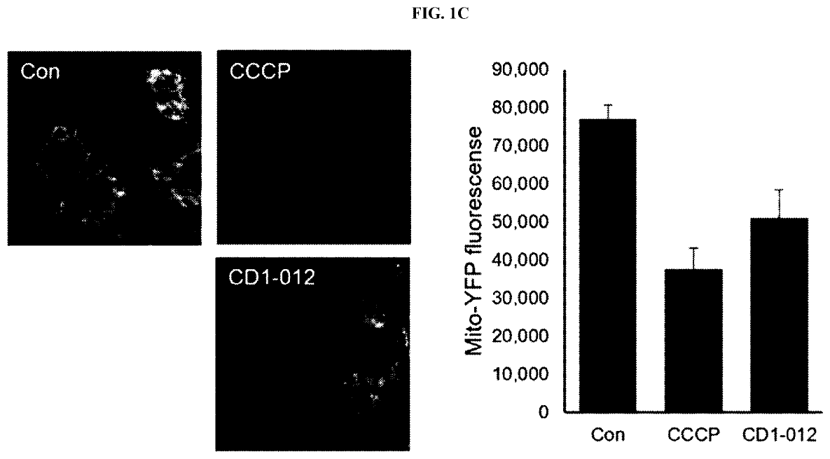

To analyze the mitophagy-promoting activity effect of the CD1-012 synthesized in the Example 1, the BEAS-2B cell line, which is a human normal lung cell line, was induced to express the mt-Keima fluorescent protein. Following this, the CD1-012 (15 μM) and the compound of Comparative Example 1, CCCP (10 μM), were treated for 24 hours. After the treatment, the mitophagy activity of each sample was measured, and the results were represented in FIGS. 1A to IC.

Specifically, FIG. 1A represents the analysis results using flow cytometry (FACS) (treatment of CD1-012 at 15 UM for 24 hours), FIG. 1B represents the analysis results using a confocal microscope (treatment of CD1-012 at 15 μM for 18 hours), and FIG. 1C represents the measurement results of the quantitative changes in the mitochondria using a fluorescent protein, mito-YFP, which includes a targeting sequence to transport protein to the mitochondria (treatment of CD1-012 at 20 μM for 24 hours). Referring R to the results, it was observed that the group treated with CD1-012 showed a significant increase in mitophagy activity compared to the untreated control group (Con). Especially in FIG. 1A, it was observed that the mitophagy activity of the sample treated with CD-012 was increased as significantly as that of the sample treated with the representative mitophagy promoting compound, CCCP.

### Example 2-2. Analysis of Mitophagy Activity Promoting Effect in Various Cell Lines

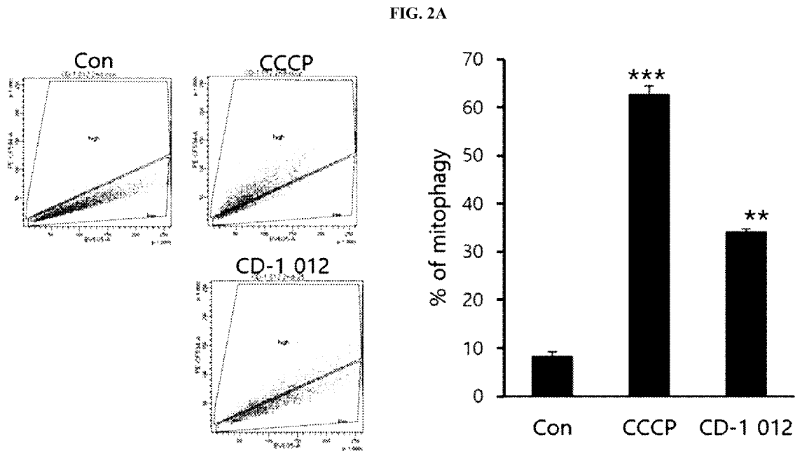

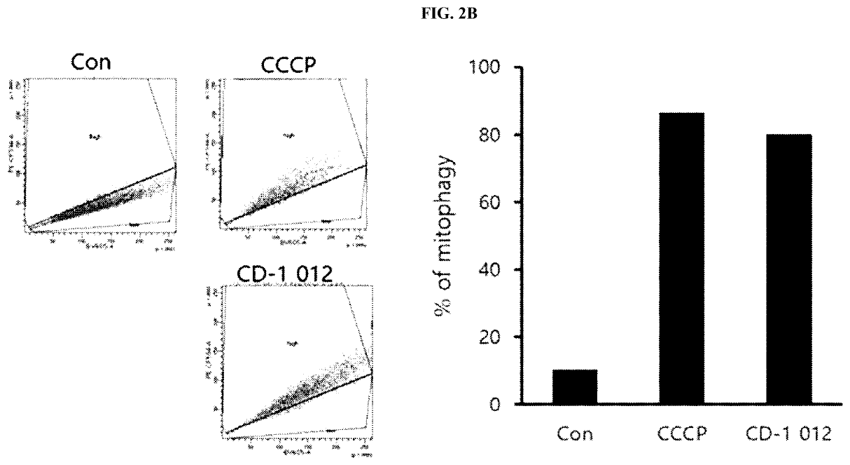

It was confirmed whether CD1-012 synthesized in Example 1 increases mitophagy activity in various cell lines. To this end, CD1-012 and Comparative Example 1 (CCCP) were treated in the SH-SY5Y cell line, a human neuroblastoma cell line expressing mt-Keima fluorescent protein, and in the HeLa-Parkin cell line, a HeLa cervical cancer cell line expressing Parkin (E3 ligase). Following this treatment, the mitophagy activity of each sample was analyzed using flow cytometry (FACS), and the results are shown in FIGS. 2A to 2B.

Specifically, FIG. 2A represents the analysis results for the SH-SY5Y cell line, and FIG. 2B represents the analysis results for the HeLa-Parkin cell line. As shown in FIGS. 2A to 2B, cells treated with the CD1-012 according to the present invention exhibited a significantly increased mitophagy activity compared to the control group (Con). These results demonstrate that the isoquinoline derivative according to the present invention can promote mitophagy activity in various cell lines.

### Example 2-3. Analysis of Mitophagy Activity Promoting Effect Depending on Treatment Concentration and Treatment Time

It was confirmed whether CD1-012 synthesized in Example 1 promotes mitophagy activity in a concentration- and time-dependent manner. To this end, the CD1-012 was treated at various concentrations or at a fixed concentration (15 μM) for different treatment durations in the BEAS-2B cell line expressing mt-Keima, and mitophagy activity was measured using a flow cytometer.

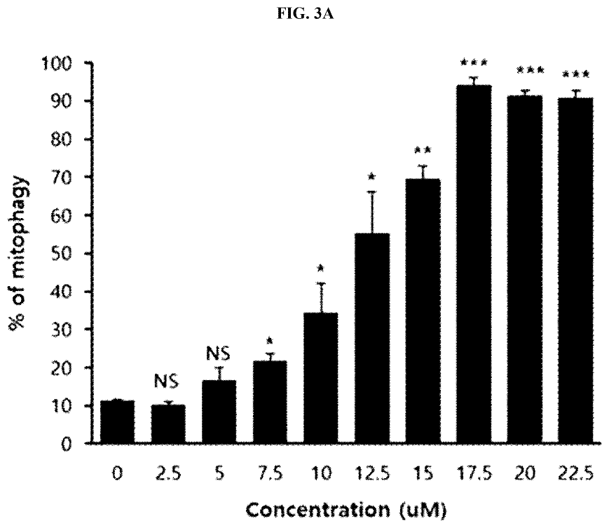

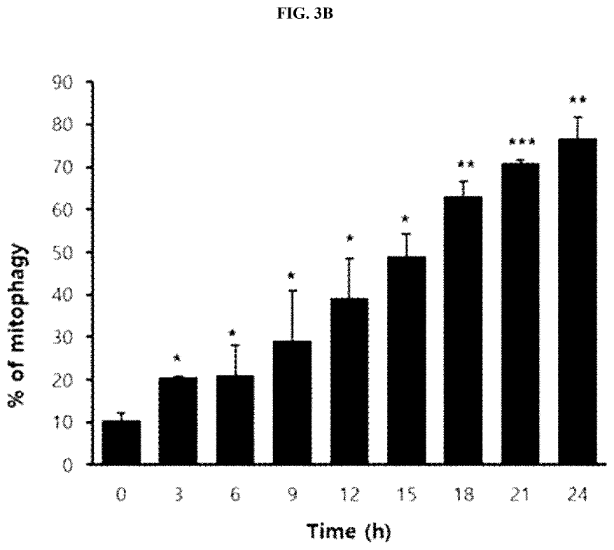

The results of mitophagy activity measurements according to the treatment concentration of CD1-012 are shown in FIG. 3A, and the results of time-dependent mitophagy activity measurements are shown in FIG. 3B. As shown in FIG. 3A, mitophagy activity began to significantly increase compared to the untreated control group when CD1-012 was treated at concentrations of 7.5 μM or higher and continued to increase in a concentration-dependent manner up to the maximum treatment concentration of 17.5 μM. Additionally, as shown in FIG. 3B, mitophagy activity began to significantly increase after 3 hours of CD1-012 treatment and reached its maximum level after 18 hours. This pattern of mitophagy activation indicates that CD1-012 directly promotes mitophagy activity in a concentration- and time-dependent manner, rather than through an indirect mechanism.

### Example 2-4. Confirmation of Specific Mitophagy Induction Activity

It was confirmed whether CD1-012 synthesized in Example 1 specifically increases only mitophagy activity. To this end, the BEAS-2B cell line expressing mt-Keima was treated with CD1-012 (15 μM) or Comparative Example 1 (CCCP, 10 μM) for 18 hours, and mitophagy activity was analyzed using a confocal microscope. Additionally, to measure autophagy activity, the BEAS-2B cell line expressing Keima fluorescent protein was incubated in Hanks' balanced salt solution (HBSS) for 3 hours to induce a starvation condition (starv.), and the results were compared with samples treated with CD1-012 (15 μM) for 18 hours using a confocal microscope.

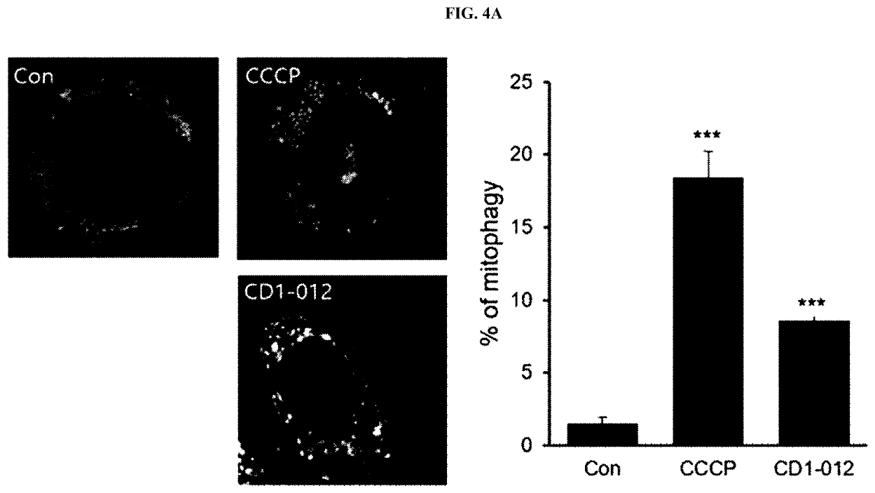

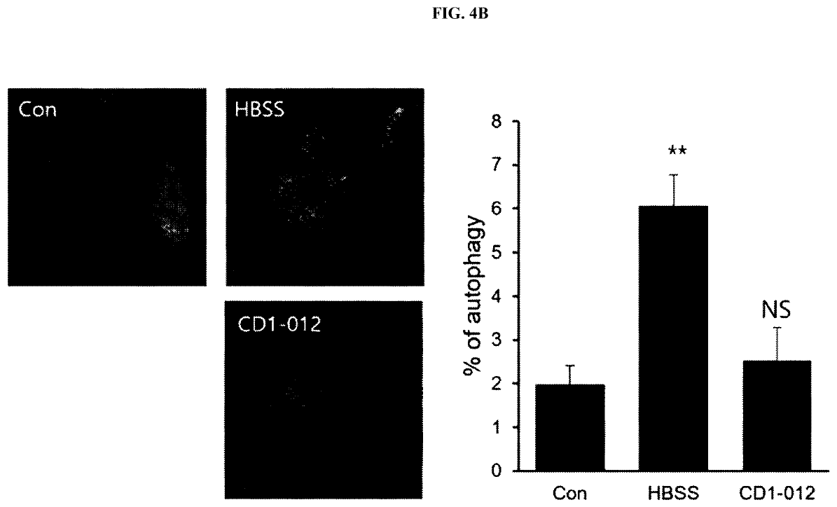

As shown in FIG. 4A, the sample treated with CD1-012 exhibited mitophagy-promoting effects similar to those observed in the CCCP-treated sample. Furthermore, as shown in FIG. 4B, the starvation-induced group exhibited a high level of autophagy, whereas the CD1-012-treated group showed no significant difference in autophagy activity compared to the untreated control group. These results suggest that the compound CD1-012 of the present invention specifically increases only mitophagy activity.

### Example 2-5. Comparison of Mitophagy Activity Induction Effects with Berberine and Palmitate

It was confirmed whether the isoquinoline derivative according to the present invention exhibits a higher mitophagy-inducing effect compared to palmatine and berberine, which are known mitophagy promoters. To this end, the human normal lung cell line BEAS-2B, expressing mt-Keima fluorescent protein, was treated with CD1-012, Comparative Example 2 (palmatine), or Comparative Example 3 (berberine) at various concentrations, and the mitophagy-inducing activity of each sample was compared.

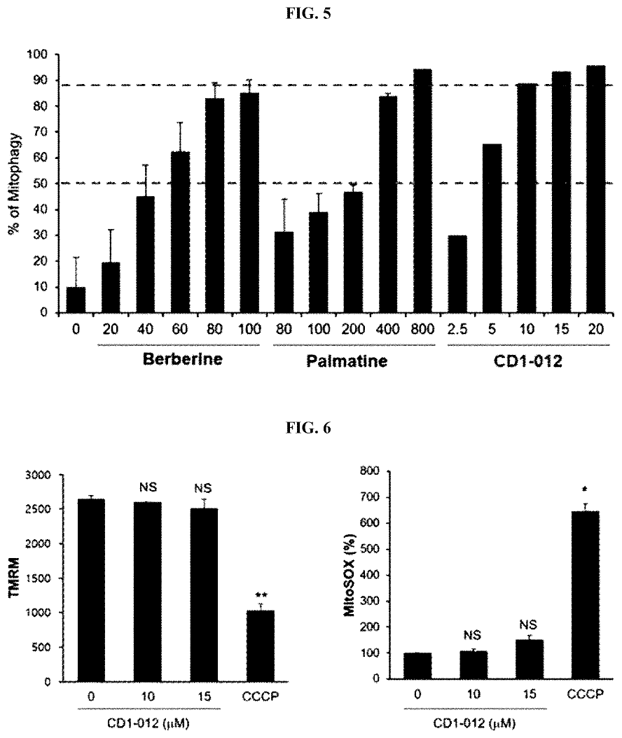

As shown in FIG. 5, mitophagy activity in BEAS-2B cells reached its maximum when treated with palmatine at 400 μM and berberine at 80 μM. However, in the CD1-012-treated group, mitophagy activity reached a comparable level at 10 μM of CD1-012 treatment. As a result, the mitophagy-inducing activity of CD1-012 was approximately 8 times higher than that of berberine and approximately 40 times higher than that of palmatine.

### Example 3. Confirmation of Induction of Mitochondrial Dysfunction of CD1-012

It was confirmed whether the isoquinoline derivative according to the present invention induces mitochondrial dysfunction, similar to CCCP. To this end, the CD1-012 (10 μM or 15 μM) and CCCP (10 μM) were treated for 24 hours, and the mitochondrial membrane potential and mitochondrial reactive oxygen species (ROS) levels in each sample were analyzed. Mitochondrial membrane potential was analyzed using the TMRM (tetramethylrhodamine methyl ester) assay, and mitochondrial ROS levels were analyzed using the MitoSOX assay.

As shown in FIG. 6, in the CCCP-treated group, mitochondrial membrane potential was significantly reduced, whereas in the CD1-012-treated group, no decrease in mitochondrial membrane potential was observed. Additionally, in the CCCP-treated group, mitochondrial ROS levels were significantly increased, while in the CD1-012-treated group, no increase in mitochondrial ROS was detected. These results indicate that, unlike CCCP, which increases mitophagy activity by inducing mitochondrial dysfunction through mitochondrial membrane potential reduction, CD1-012 does not induce mitochondrial dysfunction.

### Example 4. Confirmation of PINK1-Parkin Pathway-Independent Mitophagy Activation of mCD1-012

In this example, it was confirmed whether the mitophagy activation function of the isoquinoline derivative according to the present invention is dependent on the PINK1-Parkin pathway. To this end, after a BEAS-2B cell line, in which PINK1 was knocked down using short hairpin RNA (shRNA), was treated with CCCP (10 μM) of Comparative Example 1 or CD1-012 (15 μM) for 18 hours, the mitophagy activity of each sample was analyzed using flow cytometry (FACS).

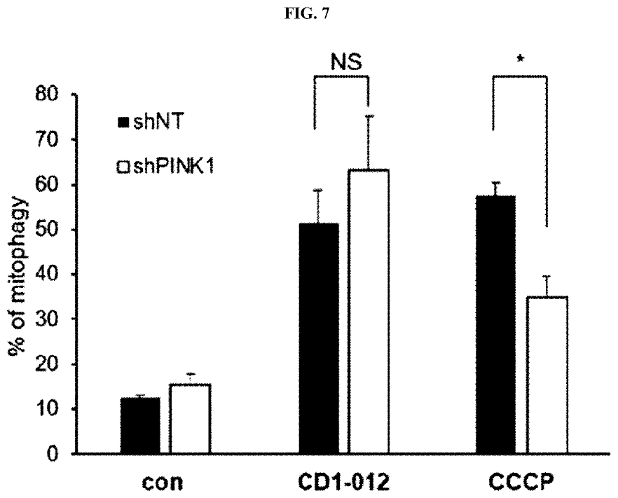

As a result, as shown in FIG. 7, the CCCP-mediated mitophagy activation in the PINK1 knockdown cell line (shPINK1) significantly decreased compared to the control cell line (shNT), whereas the CD1-012-mediated mitophagy activation did not show a significant difference compared to the untreated control. The above results demonstrate that compounds according to the present invention activate mitophagy independently of the PINK1-Parkin pathway that mediates stress-induced mitophagy.

### Example 5. Confirmation of Therapeutic Effect of CD1-012 on Peripheral Neuropathy

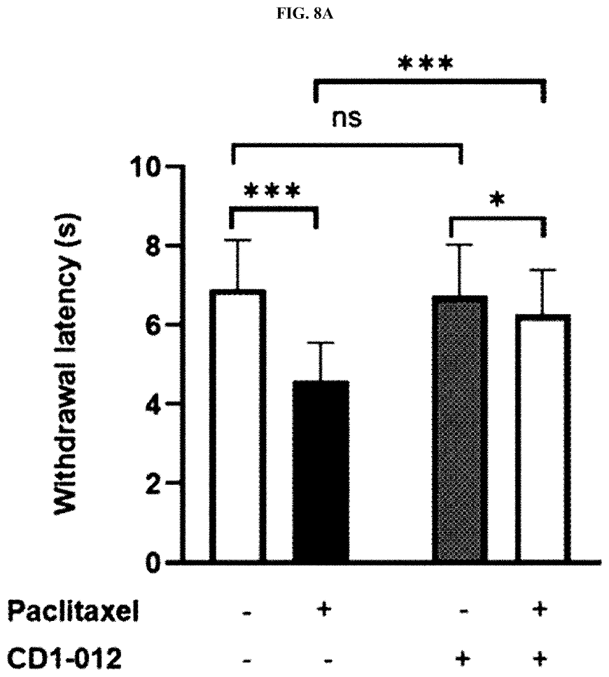

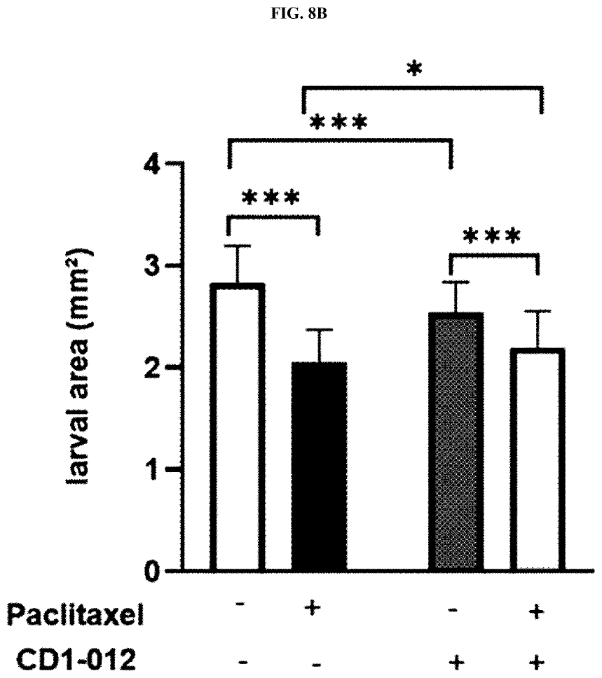

In the following example, it was confirmed whether the novel isoquinoline derivative according to the present invention exhibits a therapeutic effect on peripheral neuropathy, particularly chemotherapy-induced peripheral neuropathy. To this end, thermal sensitivity was measured in a Drosophila model of paclitaxel-induced peripheral neuropathy. According to a method reported in a recent publication (2020 PLOS One, 15 (9): e0239126. doi: 10.1371/journal.pone.0239126), third-instar larvae of Drosophila (w1118) were treated with paclitaxel at a concentration of 20 UM for 48 hours, and a heat probe at 40° C. was applied to the A4-A5 segment of the larvae to measure the withdrawal latency, which is the time taken for the larvae to exhibit a characteristic avoidance response. As a result, after treatment with paclitaxel, hyperalgesia was observed, as indicated by a 35% reduction in withdrawal latency from 6.9 seconds to 4.5 seconds (FIG. 8A). Subsequently, to confirm the therapeutic effect of CD1-012 on peripheral neuropathy, Drosophila were treated with CD1-012 at a concentration of 1 mM along with paclitaxel (20 μM). As a result, the withdrawal latency increased from 4.5 seconds to 6.3 seconds, which corresponds to 91% of the wild-type level, indicating that the reduction in withdrawal latency caused by paclitaxel treatment was restored by CD1-012 (FIG. 8A). These results indicate that CD1-012 has a therapeutic effect on hyperalgesia induced by chemotherapeutic agents.

In addition, to evaluate whether the compound according to the present invention affects the growth of Drosophila, the size of larvae was measured after treatment with paclitaxel and CD1-012. As a result, larvae treated with paclitaxel alone showed approximately a 28% reduction in size compared to the untreated control group, whereas larvae treated with CD1-012 showed only about a 10% reduction (FIG. 8B). This indicates that the isoquinoline derivative according to the present invention does not significantly inhibit larval growth.

### Example 6. Confirmation of the Effect of CD1-012 on Improving Morphological Changes in Sensory Neurons

It has been reported that the thermal hyperalgesia observed upon chemotherapeutic agent treatment in Drosophila is primarily caused by morphological changes in the dendrite arbor of Class IV da (C4da) sensory neurons (2020 PLOS One, 15 (9): e0239126. doi: 10.1371/journal.pone.0239126; 2018 Dis Model Mech. 11 (6): dmm032938. doi: 10.1242/dmm.032938). According to these studies, chemotherapeutic agents such as paclitaxel induce an increase in the dendritic branch length and number of C4da sensory neurons, which leads to hyperalgesia. Accordingly, in this example, it was confirmed whether the isoquinoline compound according to the present invention can suppress the morphological changes in sensory neurons induced by chemotherapeutic agents.

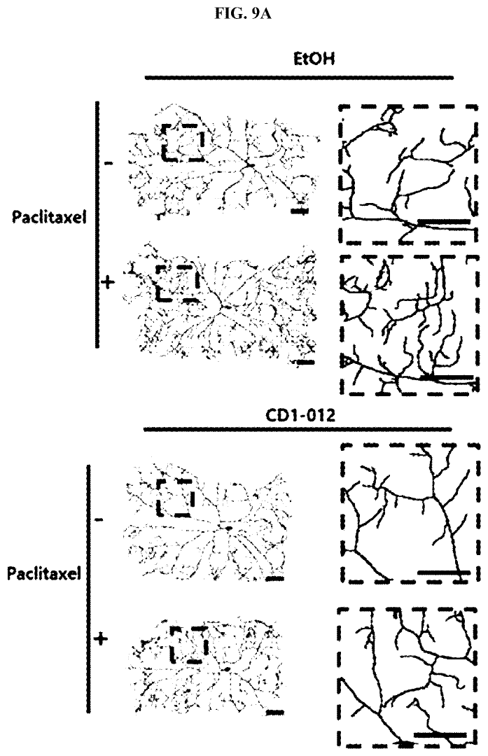

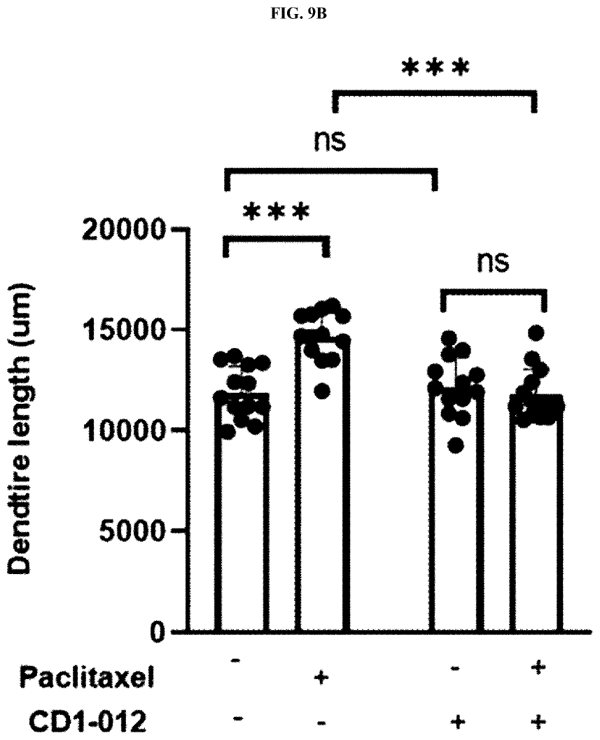

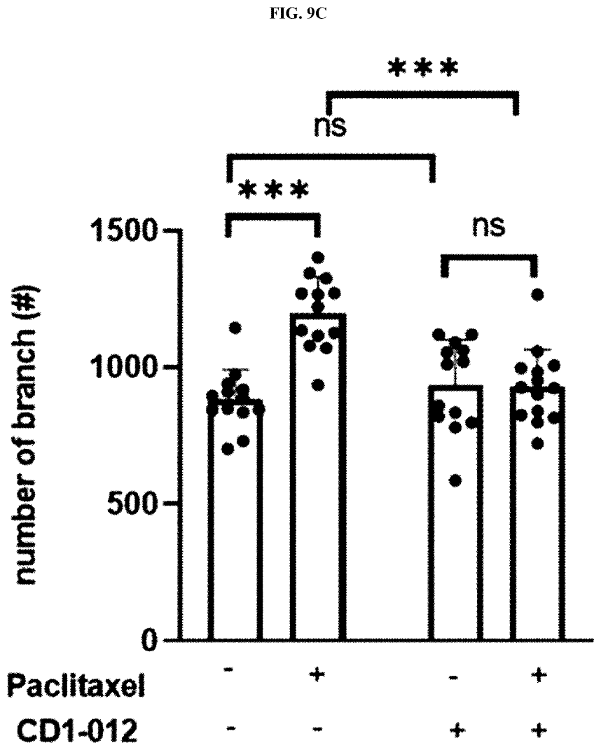

To analyze the morphological changes in the dendritic branches of C4da sensory neurons, transgenic Drosophila expressing the fluorescent protein CD4-tdTomato specifically in C4da neurons (ppk^{1a}>CD4-tdTom) was generated. After treatment of the transgenic flies with paclitaxel and CD1-012, morphological changes in the dendritic branches of C4da sensory neurons were examined. As a result, treatment with paclitaxel increased the total dendrite length and the number of dendritic branches by 22% and 33%, respectively, confirming that morphological changes in the sensory neurons were induced. In contrast, when CD1-012 was co-administered, the paclitaxel-induced increases in dendrite length and number of branches were significantly suppressed (FIGS. 9A to 9C). These results demonstrate that the compound according to the present invention can suppress degeneration of C4da sensory neurons induced by chemotherapeutic agents, thereby improving and treating hyperalgesia.

### Example 7. Confirmation of ATG5- and ATG7-Independent Therapeutic Effect of CD1-012 on Peripheral Neuropathy

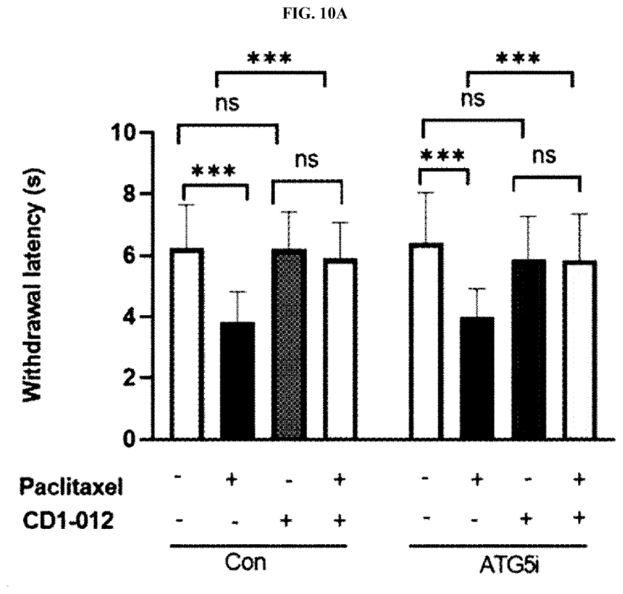

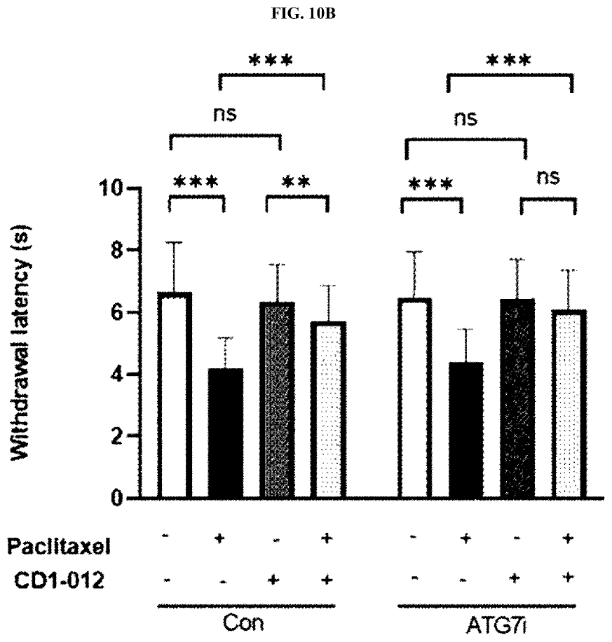

In this example, an experiment was conducted to verify the therapeutic mechanism of the compound according to the present invention on peripheral neuropathy, particularly chemotherapy-induced peripheral neuropathy. To this end, the therapeutic effect of CD1-012 on peripheral neuropathy was evaluated using animal models in which the expression of ATG5 or ATG7, essential genes in the canonical mitophagy pathway, the most representative mitophagy regulatory pathway, was suppressed. Specifically, paclitaxel (20 μM) and CD1-012 (1 mM) were co-administered to Drosophila expressing shATG7 or shATG5 in C4da sensory neurons, as well as to control flies (wild-type), and a heat probe assay was conducted using a 40° C. thermal probe. As a result, the therapeutic effect of CD1-012 on chemotherapy-induced peripheral neuropathy was similarly observed even in Drosophila in which the expression of ATG5 or ATG7 was suppressed, thereby blocking the mitophagy pathway (FIGS. 10A and 10B). These results indicate that the compound according to the present invention exerts a therapeutic effect on peripheral neuropathy through a mechanism that is independent of the canonical mitophagy pathway.

### Example 8. Confirmation of ULK1 and Rab9 Dependency in the Therapeutic Effect of CD1-012 on Peripheral Neuropathy

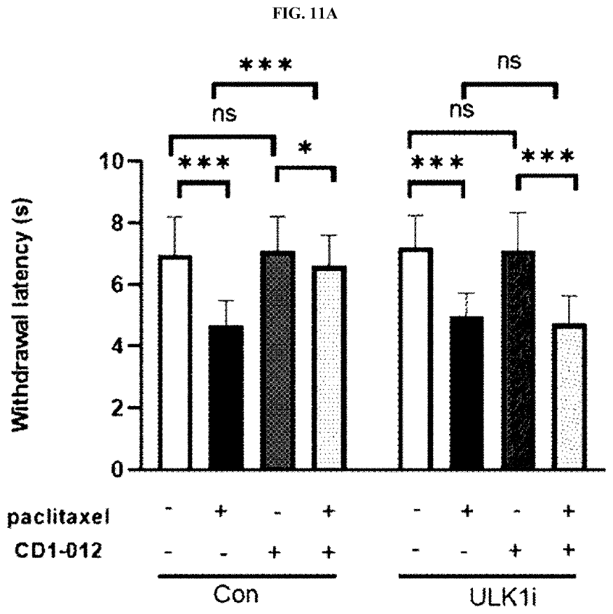

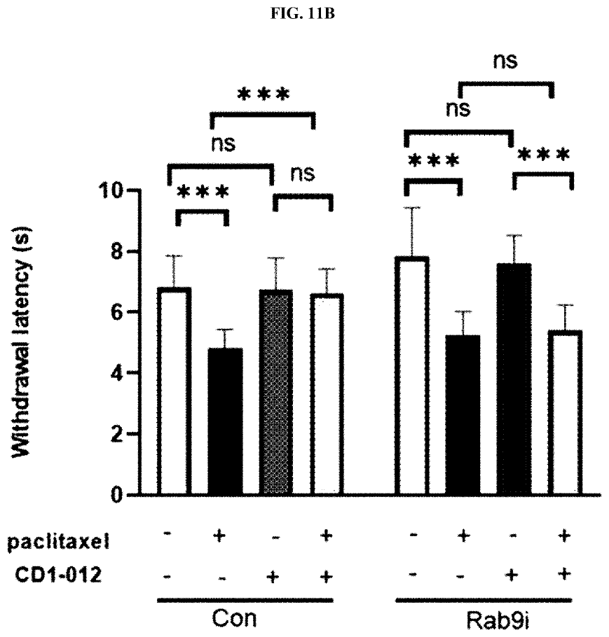

Subsequently, to understand the molecular mechanism underlying the therapeutic effect of CD1-012 on chemotherapy-induced peripheral neuropathy, the therapeutic effect of CD1-012 was examined after suppressing the expression of ULK1 or Rap9, which are essential genes in the recently identified alternative mitophagy pathway. To this end, a heat probe assay was performed using a 40° C. thermal probe after co-treating paclitaxel (20 μM) and CD1-012 (1 mM) in Drosophila expressing shULK1 or shRap9 in C4da sensory neurons, as well as in control Drosophila (wild-type). As a result, in the control group, the peripheral neuropathy symptoms (reduction in withdrawal latency) induced by paclitaxel were recovered by CD1-012 treatment. In contrast, in Drosophila with suppressed expression of ULK1 or Rap9, the therapeutic effect of CD1-012 on paclitaxel-induced peripheral neuropathy was completely abolished (FIGS. 11A and 11B). These results indicate that CD1-012 exerts a therapeutic effect on peripheral neuropathy by promoting mitophagy through the alternative mitophagy pathway.

### Example 9. Confirmation of the Therapeutic Effect of CD1-012 on Peripheral Neuropathy Using a Mouse Model

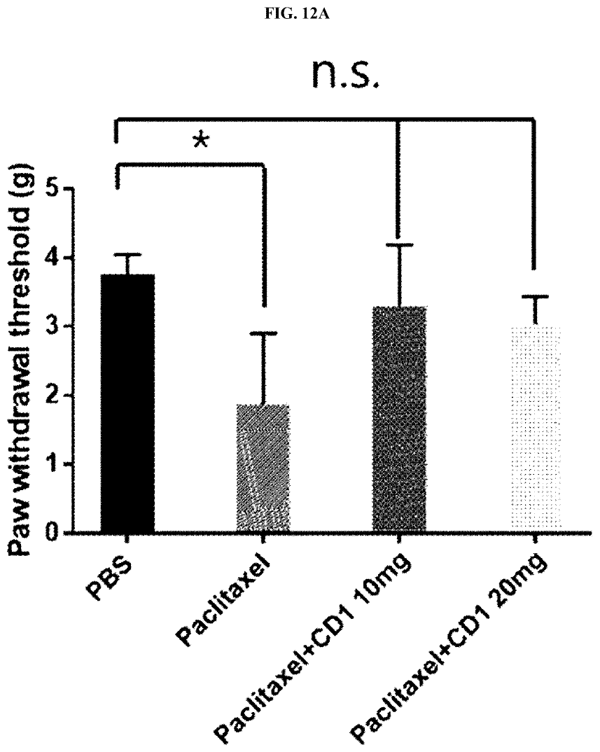

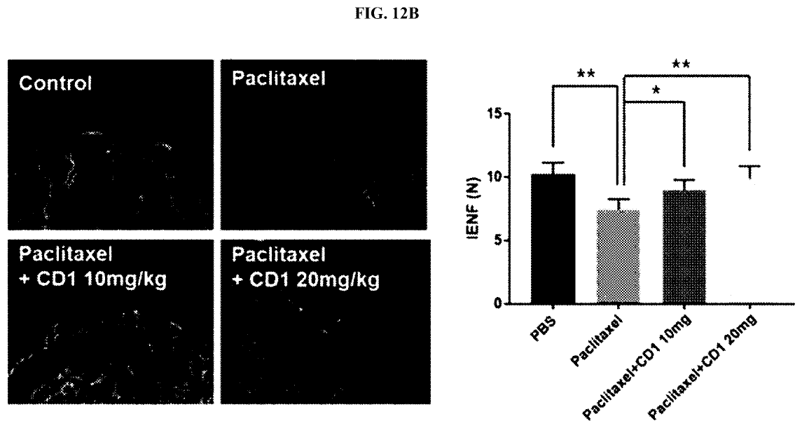

To more clearly verify the therapeutic effect of the isoquinoline derivative of the present invention on peripheral neuropathy, particularly chemotherapy-induced peripheral neuropathy, experiments were conducted using a peripheral neuropathy mouse model. Specifically, peripheral neuropathy was induced in mice by administration of a chemotherapeutic agent (paclitaxel), and CD1-012 was co-administered at a dose of 10 mg/kg or 20 mg/kg. Sensitivity to pain was then analyzed using the vonfrey hair test. As a result, the chemotherapy-induced peripheral neuropathy mouse model exhibited increased pain sensitivity, whereas the mice co-administered with CD1-012 showed a significant improvement in such hyperalgesic symptoms (FIG. 12A). In addition, examination of nerve distribution in the footpad revealed that the number of peripheral nerves (intraepidermal nerve fibers: IENFs) was reduced in the chemotherapy-induced peripheral neuropathy mouse model treated with paclitaxel, whereas the mice treated with CD1-012 showed recovery to normal levels (FIG. 12B). These results clearly demonstrate that the compound according to the present invention can suppress neurodegeneration and improve hyperalgesia not only in a peripheral neuropathy Drosophila model but also in a mouse model.

As demonstrated in the above examples, the present inventors confirmed that the isoquinoline compound CD1-012, which has mitophagy-specific promoting activity, exhibits a therapeutic effect on peripheral neuropathy, particularly chemotherapy-induced peripheral neuropathy. The compound of the present invention may improve hyperalgesia, a representative symptom of peripheral neuropathy, and effectively suppress degeneration of sensory neurons and reduction in the distribution of peripheral nerves. In addition, the excellent therapeutic effect of CD1-012 on peripheral neuropathy was shown to depend on the alternative mitophagy pathway mediated by ULK1 and Rab9, while being independent of the canonical mitophagy pathway mediated by ATG5 and ATG7. Therefore, by considering the underlying molecular mechanism, peripheral neuropathy may be treated across various patient populations through appropriate use. Accordingly, the novel isoquinoline derivative according to the present invention is expected to be usefully applicable for the prevention and treatment of peripheral neuropathy as a compound capable of fundamentally treating peripheral neuropathy, including chemotherapy-induced peripheral neuropathy.

The foregoing description of the present invention is provided by way of example, and those skilled in the art to which the present invention pertains will understand that various modifications can be easily made in other specific forms without altering the technical spirit or essential characteristics of the present invention. Therefore, the embodiments described above should be understood as illustrative in all respects and not as limiting.

### Manufacturing Example of Drug

The active material according to the present invention may be formulated in various forms depending on its purpose. The following are some methods of formulating the active material according to the present invention comprising the active ingredient, and the present invention is not limited thereto.

### <Manufacturing Example of Drug 1> Manufacturing of Acid Formulation

Active material 2 g
Lactose 1 g

The ingredients were mixed and filled in a sealed pack to prepare the acid formulation.

### <Manufacturing Example of Drug 2> Manufacturing of Tablet

Active material 100 mg
Cornstarch 100 mg
Lactose 100 mg
Magnesium stearate 2 mg

The ingredients were mixed and then tableted according to conventional tablet manufacturing methods to prepare the tablet.

### <Manufacturing Example of Drug 3> Manufacturing of Capsule

Active material 100 mg
Cornstarch 100 mg
Lactose 100 mg
Magnesium stearate 2 mg

The ingredients were mixed and then filled into gelatin capsules according to conventional capsule manufacturing methods to prepare the capsule formulation.

### <Manufacturing Example of Drug 4> Manufacturing of Injectable

Active material 10 g/mL
Diluted hydrochloric acid BP until pH 3.5
Injectable sodium chloride BP up to 1 mL

The active material according to the present invention was dissolved in an appropriate volume of injectable sodium chloride BP, and the pH of the resulting solution was adjusted to pH 3.5 using diluted hydrochloric acid BP. The volume was adjusted using injectable sodium chloride BP and mixed thoroughly. The solution was filled into transparent glass 5 mL type I ampoules, sealed under an air upper grid by melting the glass, and sterilized by autoclaving at 120° C. for at least 15 minutes to prepare the injectable.

### <Manufacturing Example of Drug 5> Manufacturing of Nasal Spray

Active material 1.0 g
Sodium acetate 0.3 g
Methylparaben 0.1 g
Propylparaben 0.02 g
Sodium chloride appropriate amount
HCl or NaOH for pH adjustment appropriate amount
Purified water appropriate amount

According to conventional nasal spray manufacturing methods, it was prepared to contain 3 mg of active material per 1 mL of saline (0.9% NaCl, w/v, solvent is purified water). It was filled into an opaque spray container and sterilized to prepare the nasal spray.

### <Manufacturing Example of Drug 6> Manufacturing of Liquid Formulation

Active material 100 mg
High-fructose corn syrup 10 g
Mannitol 5 g
Purified water appropriate amount

Each ingredient was dissolved in purified water according to conventional liquid formulation manufacturing methods, lemon flavor was added, and then the ingredients were mixed. Purified water was added to adjust the total to 100 mL, and it was filled into a brown bottle and sterilized to prepare the liquid formulation.

### Manufacturing Example of Health Food

The active material according to the present invention may be manufactured into various forms of health food depending on its purpose. The following are examples of manufacturing health foods comprising an active material as an active ingredient according to the present invention, but the present invention is not limited thereto.

### <Manufacturing Example of Health Food 1> Manufacturing of Dairy Products

weight part of the active material of the present invention was added to milk, and various dairy products such as butter and ice cream were manufactured using the milk.

### <Manufacturing Example of Health Food 2> Manufacturing of Seonsik

Brown rice, barley, glutinous rice, and barnyard millet were gelatinized by a known method and dried, and then mixed and ground into a powder of 60 mesh. Black beans, black sesame, and perilla seeds were also steamed by a known method, dried, and then mixed and ground into a powder of 60 mesh. The active material of the present invention was concentrated under reduced pressure in a vacuum concentrator, and a dried powder was obtained. The following proportions of the manufactured cereals, seeds, and dried powder of the active material were mixed.
Cereals (brown rice 34 weight parts, barnyard millet 19 weight parts, barley 20 weight parts),
Seeds (perilla seeds 7 weight parts, black beans 8 weight parts, black sesame 7 weight parts),
Active material (2 weight parts),
Ganoderma lucidum (1.5 weight parts), and
Rehmannia glutinosa (1.5 weight parts).

### Manufacturing Example of Health Functional Food Composition

Active materials according to the present invention may be manufactured into various forms of health functional food compositions depending on its purpose. The following are examples of manufacturing health functional food compositions comprising an active material as an active ingredient according to the present invention, but the present invention is not limited thereto.

### <Manufacturing Example of Health Functional Food Composition 1> Manufacturing of Health Functional Food Composition

Active material 100 mg
Vitamin mixture appropriate amount
Vitamin A acetate 70 μg
Vitamin E 1.0 mg
Vitamin B1 0.13 mg
Vitamin B2 0.15 mg
Vitamin B6 0.5 mg
Vitamin B12 0.2μ g
Vitamin C 10 mg
Biotin 10 μg
Nicotinamide 1.7 mg
Folic acid 50 μg
Pantothenic acid calcium 0.5 mg
Mineral mixture appropriate amount
Ferrous sulfate 1.75 mg
Zinc oxide 0.82 mg
Magnesium carbonate 25.3 mg
Potassium dihydrogen phosphate 15 mg
Calcium hydrogen phosphate 55 mg
Potassium citrate 90 mg
Calcium carbonate 100 mg
Magnesium chloride 24.8 mg

The composition ratio of the vitamins and minerals mixture was mixed as a desirable example suitable for relatively health functional foods, but it is acceptable to modify the mixing ratio at will. The above ingredients were mixed and then granulated, and may be used to manufacture the health functional food composition according to a conventional method.

### <Manufacturing Example of Health Functional Food Composition 2> Manufacturing of Health Functional Beverage

Active material 100 mg
Citric acid 100 mg
Oligosaccharide 100 mg
Plum concentrate 2 mg
Taurine 100 mg
Add purified water to make a total of 500 mL

The above ingredients were mixed and then heated with stirring at 85° C. for about 1 hour. The resulting solution was filtered, filled into a sterilized container, and sterilized by sealing. After refrigeration, it may be used to manufacture the health beverage composition of the present invention. The composition ratio was mixed as a desirable example suitable for relatively preferred beverages, but it may be modified according to regional and ethnic preferences, demand groups, demand countries, and usage purposes.

The foregoing description of the present invention is provided by way of example, and those skilled in the art to which the present invention pertains will understand that various modifications can be easily made in other specific forms without departing from the technical spirit or essential characteristics of the present invention. Therefore, the embodiments described above should be understood as illustrative in all respects and not as limiting.

### INDUSTRIAL APPLICABILITY

The present invention relates to a pharmaceutical composition for preventing or treating peripheral neuropathy, and the like. The present invention has been completed by confirming that an isoquinoline derivative discovered through a screening based on mitophage activity exhibits an excellent mitophage promoting effect, and thus, the isoquinoline derivative can be utilized as a fundamental therapeutic agent for peripheral neuropathy. Specifically, it has been found that the isoquinoline derivative according to the present invention treats hyperalgesia in an animal model of anticancer drug-inducible peripheral neuropathy, also suppresses morphological changes of sensory nerves, which have been identified as a key cause of peripheral neuropathy, and can increase the distribution and number of peripheral nerves. Therefore, the isoquinoline derivative according to the present invention is a fundamental therapeutic agent capable of suppressing the main symptoms and causes of peripheral neuropathy, particularly, anticancer drug-induced peripheral neuropathy, and is expected to be usefully utilized in the field of prevention, amelioration, and/or treatment of the disease.

1. A method of preventing or treating peripheral neuropathy, comprising: administering a pharmaceutical composition comprising an isoquinoline derivative represented by Chemical Formula 1 below or a pharmaceutically acceptable salt thereof as an active ingredient to a subject in need thereof

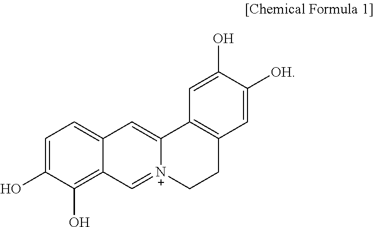 [SMILES: Oc1cc2c(cc1O)-c1cc3ccc(O)c(O)c3c[n+]1CC2]

2. The method of claim 1, wherein the isoquinoline derivative or pharmaceutically acceptable salt thereof specifically promotes mitophagy activity.

3. The method of claim 2, wherein the mitophagy activity is independent of one or more selected from the group consisting of PINK1, ATG5, and ATG7.

4. The method of claim 2, wherein the mitophagy activity is dependent on ULK1 or Rab9.

5. The method of claim 1, wherein the isoquinoline derivative or a pharmaceutically acceptable salt thereof inhibits morphological degeneration of sensory nerves.

6. The method of claim 1, wherein the isoquinoline derivative or a pharmaceutically acceptable salt thereof increases the number of peripheral nerves.

7. The method of claim 1, wherein the peripheral neuropathy is chemotherapy-induced peripheral neuropathy.

8. The method of claim 7, wherein the chemotherapeutic agent is one selected from the group consisting of paclitaxel, bortezomib, oxaliplatin, vincristine, cisplatin, taxol, docetaxel, ixabepilone, thalidomide, Velcade, and lenalidomide.

9. The method of claim 1, wherein the isoquinoline derivative or a pharmaceutically acceptable salt thereof is present in an amount of greater than 0 and up to 40 μM.

10. The method of claim 1, wherein the isoquinoline derivative or a pharmaceutically acceptable salt thereof improves mitochondrial dysfunction.

11. The method of claim 10, wherein the improvement in mitochondrial dysfunction is one selected from the group consisting of:
(a) maintaining the normal membrane potential level in mitochondria;
(b) reducing the reactive oxygen species (ROS) level in mitochondria; and
(c) increasing the ATP synthesis capacity of mitochondria.

12. The method of any one of claims 1 to 11, wherein the method comprises using a kit comprising the pharmaceutical composition, and instructions.

13. A food composition for method of preventing or improving peripheral neuropathy, comprising administering a food composition comprising an isoquinoline derivative represented by Chemical Formula 1 below or a sitologically acceptable salt thereof as an active ingredient to a subject in need thereof

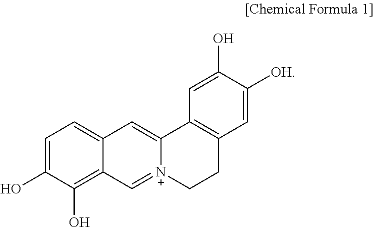 [SMILES: Oc1cc2c(cc1O)-c1cc3ccc(O)c(O)c3c[n+]1CC2]

14. The method of claim 13, wherein the peripheral neuropathy is chemotherapy-induced peripheral neuropathy.

15-17. (canceled)
